(19) United States
(12) Patent Application Publication
(10) Pub. No.: US 2023/0220320 A1
(43) Pub. Date: Jul. 13, 2023

(54) Method For Capturing And Purification Of Biologics

(71) Applicant: Amicus Therapeutics, Inc., Philadelphia, PA (US)

(72) Inventors: Kumar Dhanasekharan, Cranbury, NJ (US); Christine Caroselli, Cranbury, NJ (US); Paul Langevin, Ayer, MA (US)

(73) Assignee: Amicus Therapeutics, Inc., Philadelphia, PA (US)

(21) Appl. No.: 17/640,456

(22) PCT Filed: Sep. 4, 2020
(86) PCT No.: PCT/US2020/049535

§ 371 Date: Mar. 4, 2022

Related U.S. Application Data
(60) Provisional application No. 62/897,018, filed on Sep. 6, 2019.

(51) Int. Cl. C12M 1/00; C12M 1/12

(52) U.S. Cl. CPC C12M 23/58; C12M 47/12; C12M 37/02; C12M 23/02

(57) ABSTRACT

Methods for the continuous production, capturing and purification of biologics such as recombinant proteins are described. Also described are pharmaceutical compositions comprising such biologics, as well as methods of treatment and uses of such biologics.

### TECHNICAL FIELD

Principles and embodiments of the present invention relate generally to the manufacturing of biologics, particularly lysosomal enzymes that have a high content of mannose-6-phosphate.

### BACKGROUND

Lysosomal storage disorders are a group of autosomal recessive genetic diseases characterized by the accumulation of molecular substrates such as glycosphingolipids, glycogen, or mucopolysaccharides within intracellular compartments called lysosomes. Individuals with these diseases carry mutant genes coding for enzymes which are defective in catalyzing the hydrolysis of one or more of these substrates, which then build up in the lysosomes. For example, Pompe disease, also known as acid maltase deficiency or glycogen storage disease type II, is one of several lysosomal storage disorders. Other examples of lysosomal disorders include Gaucher disease, GM1-gangliosidosis, fucosidosis, mucopolysaccharidoses, Hurler-Scheie disease, Niemann-Pick A and B diseases, and Fabry disease. Pompe disease is also classified as a neuromuscular disease or a metabolic myopathy.

Pompe disease is estimated to occur in about 1 in 40,000 births, and is caused by a mutation in the GAA gene, which codes for the enzyme lysosomal α-glucosidase (EC:3.2.1.20), also commonly known as acid a-glucosidase. Acid α-glucosidase is involved in the metabolism of glycogen, a branched polysaccharide which is the major storage form of glucose in animals, by catalyzing its hydrolysis into glucose within the lysosomes. Because individuals with Pompe disease produce mutant, defective acid α-glucosidase which is inactive or has reduced activity, glycogen breakdown occurs slowly or not at all, and glycogen accumulates in the lysosomes of various tissues, particularly in striated muscles, leading to a broad spectrum of clinical manifestations, including progressive muscle weakness and respiratory insufficiency. Tissues such as the heart and skeletal muscles are particularly affected.

Recent treatment options for lysosomal storage disorders include enzyme replacement therapy (ERT) with recombinant enzymes. For example, one treatment option for Pompe disease includes ERT with recombinant human acid α-glucosidase (rhGAA). Conventional rhGAA products are known under the names alglucosidase alfa, Myozyme® or Lumizyme®, from Genzyme, Inc. ERT is a chronic treatment required throughout the lifetime of the patient, and involves administering the replacement enzyme by intravenous infusion. The replacement enzyme is then transported in the circulation and enters lysosomes within cells, where it acts to break down the accumulated substrate (e.g. glycogen), compensating for the deficient activity of the endogenous defective mutant enzyme, and thus relieving the disease symptoms. In subjects with infantile onset Pompe disease, treatment with alglucosidase alfa has been shown to significantly improve survival compared to historical controls, and in late onset Pompe disease, alglucosidase alfa has been shown to have a statistically significant, if modest, effect on the 6-Minute Walk Test (6MWT) and forced vital capacity (FVC) compared to placebo.

However, the majority of subjects either remain stable or continue to deteriorate while undergoing treatment with alglucosidase alfa. The reason for the apparent sub-optimal effect of ERT with alglucosidase alfa is unclear, but could be partly due to the progressive nature of underlying muscle pathology, or the poor tissue targeting of the current ERT. For example, the infused enzyme is not stable at neutral pH, including at the pH of plasma (about pH 7.4), and can be irreversibly inactivated within the circulation. Furthermore, infused alglucosidase alfa shows insufficient uptake in key disease-relevant muscles, possibly due to inadequate glycosylation with mannose-6-phosphate (M6P) residues. Such residues bind cation-independent mannose-6-phosphate receptors (CIMPR) at the cell surface, allowing the enzyme to enter the cell and the lysosomes within. Therefore, high doses of the enzyme may be required for effective treatment so that an adequate amount of active enzyme can reach the lysosomes, making the therapy costly and time-consuming.

There are seven potential N-linked glycosylation sites on rhGAA. Since each glycosylation site is heterogeneous in the type of N-linked oligosaccharides (N-glycans) present, rhGAA consist of a complex mixture of proteins with N-glycans having varying binding affinities for M6P receptor and other carbohydrate receptors. rhGAA that contains a high mannose N-glycans having one M6P group (mono-M6P) binds to CIMPR with low (˜7,000 nM) affinity while rhGAA that contains two M6P groups on same N-glycan (bis-M6P) bind with high (˜2 nM) affinity. Representative structures for non-phosphorylated, mono-M6P, and bis-M6P glycans are shown by FIG. 1A. The mannose-6-P group is shown by FIG. 1B. Once inside the lysosome, rhGAA can enzymatically degrade accumulated glycogen. However, conventional rhGAAs have low total levels of M6P- and bis-M6P bearing glycans and, thus, target muscle cells poorly resulting in inferior delivery of rhGAA to the lysosomes. Productive drug targeting of rhGAA is shown in FIG. 2A. The majority of rhGAA molecules in these conventional products do not have phosphorylated N-glycans, thereby lacking affinity for the CIMPR. Non-phosphorylated high mannose glycans can also be cleared by the mannose receptor which results in non-productive clearance of the ERT (FIG. 2B).

The other type of N-glycans, complex carbohydrates, which contain galactose and sialic acids, are also present on rhGAA. Since complex N-glycans are not phosphorylated they have no affinity for CIMPR. However, complex-type N-glycans with exposed galactose residues have moderate to high affinity for the asialoglycoprotein receptor on liver hepatocytes which leads to rapid non-productive clearance of rhGAA (FIG. 2B).

Current manufacturing processes used to make conventional rhGAA, such as Myozyme®, Lumizyme® or alglucosidase alfa, have not significantly increased the content of M6P or bis-M6P because cellular carbohydrate processing is naturally complex and extremely difficult to manipulate. Accordingly, there remains a need for further improvements to enzyme replacement therapy for treatment of Pompe disease, such as new manufacturing, capturing and purification processes for rhGAA.

Similarly, other recombinant proteins that are targeted to the lysosome, such as other lysosomal enzymes, also bind CIMPR. However, current manufacturing processes used to make other conventional recombinant proteins that are targeted to lysosomes do not provide recombinant proteins with a high content of M6P or bis-M6P. Accordingly, there remains a need for further improvements in the manufacturing, capturing and purification processes for these other recombinant proteins as well.

### SUMMARY

One aspect of the present invention is related to a method for producing biologics. In various embodiments of this aspect, the method comprises culturing host cells in a bioreactor and loading biologics-containing fluid (e.g. filtrate) onto at least two capture columns, wherein the at least two capture columns have a total capture column volume, and wherein the ratio of the bioreactor volume to the total capture column volume is in the range of about 500:1 to about 10:1, such as ratios of about 100:1 to about 20:1. In various embodiments of this aspect, the total capture column residence time (i.e. the quotient of the total capture column volume and the volumetric flow rate loading the capture columns) is in the range of about 0.5 minutes to 200 minutes, such as about 10 to 70 minutes.

In one or more embodiments, the method comprises: culturing host cells in a bioreactor that produce and optionally secrete biologics; removing media and/or cell suspension from the bioreactor; processing the media and/or cell suspension to separate a filtrate containing the biologics; loading the filtrate onto at least two capture columns to capture the biologics; eluting a first biologic product from the at least two capture columns; loading the first biologic product onto one or more purification columns; and eluting a second biologic product from the one or more purification columns; wherein the bioreactor has a bioreactor volume, the at least two capture columns have a total capture column volume, and wherein the ratio of the bioreactor volume to the total capture column volume is in the range of about 500:1 to about 10:1, such as ratios of about 100:1 to about 20:1. Alternatively, in one or more embodiments, the biologics are not secreted and are removed after lysing cells.

In one or more embodiments, the biologic comprises one or more of a recombinant protein, a virus particle or an antibody.

In one or more embodiments, the recombinant protein is a secreted protein, a membrane protein or an intracellular protein produced by the host cells. In one or more embodiments, the recombinant protein is separated into the filtrate from cells and/or cellular organelles. In one or more embodiments, the filtrate is separated by filtration or centrifugation.

In one or more embodiments, the at least two capture columns are loaded sequentially to provide continuous loading of the filtrate onto the at least two capture columns.

In one or more embodiments, the filtrate is loaded on the at least two capture columns at a filtrate load rate in the range of about 0.5 to about 100 column volumes (CV) per hour, such as about 1 to about 40 CV per hour.

In one or more embodiments, the filtrate is loaded on the at least two capture columns to provide a capture column load time of less than 48 hours for each capture column, such as less than 24 hours.

In one or more embodiments, the biologics comprises recombination human lysosomal protein.

In one or more embodiments, the at least two capture columns comprise at least two anion exchange chromatography (AEX) columns. In one or more embodiments, the at least two capture columns comprise at least two affinity chromatography columns. The affinity chromatography columns may be one or more of a protein A column and protein Z column. In one or more embodiments, the at least two capture columns comprise at least two cation exchange chromatography (CEX) columns. In one or more embodiments, the at least two capture columns comprise at least two immobilized metal affinity chromatography (IMAC) columns. In one or more embodiments, the at least two capture columns comprise at least two size exclusion chromatography columns. In one or more embodiments, the at least two capture columns comprise at least two hydrophobic interaction chromatography (HIC) columns.

In one or more embodiments, the one or more purification columns comprise one or more anion exchange chromatography (AEX) columns. In one or more embodiments, the one or more purification columns comprise one or more affinity chromatography columns. The affinity chromatography columns may be one or more of a protein A column and protein Z column. In one or more embodiments, the one or more purification columns comprise one or more cation exchange chromatography (CEX) columns. In one or more embodiments, the one or more purification columns comprise one or more immobilized metal affinity chromatography (IMAC) columns. In one or more embodiments, the one or more purification columns comprise one or more size exclusion chromatography columns. In one or more embodiments, the one or more purification columns comprise one or more hydrophobic interaction chromatography (HIC) columns. In one or more embodiments, the one or more purification columns comprise one or more immobilized metal affinity chromatography (IMAC) columns.

In one or more embodiments, the second biologic product is eluted from the one or more purification columns within 48 hours of removing the media and/or cell suspension from the bioreactor.

In one or more embodiments, the one or more purification columns have a total purification column volume and the ratio of the bioreactor volume to the total purification column volume is in the range of about 5,000:1 to about 50:1.

In one or more embodiments, the ratio of the total capture column volume to the total purification column volume is in the range of about 20:1 to about 1:1.

Another aspect of the disclosure describes a method for manufacturing recombinant human lysosomal proteins. In some embodiments, the method comprises: culturing host cells in a bioreactor that produce a recombinant human lysosomal protein, removing media and/or cell suspension from the bioreactor, processing the media and/or cell suspension to separate a filtrate containing the lysosomal protein, loading the filtrate onto at least two anion exchange chromatography (AEX) columns to capture the lysosomal protein, eluting a first biologic product from the at least two AEX columns, loading the first biologic product onto one or more immobilized metal affinity chromatography (IMAC) columns, and eluting a second biologic product from the one or more IMAC columns, wherein the bioreactor has a bioreactor volume, the at least two AEX columns have a total AEX column volume, and wherein the ratio of the bioreactor volume to the total AEX column volume is in the range of about 500:1 to about 10:1, such as ratios of about 100:1 to about 20:1.

In one or more embodiments, the lysosomal protein is a secreted protein, a membrane protein or an intracellular protein produced by the host cells. In some embodiments, the intracellular protein is separated into the filtrate by lysing the cells to prepare a cell lysate. In some embodiments, the cell lysate is separated from the filtrate by a filtration or a centrifugation.

In some embodiments, the at least two AEX columns are loaded sequentially to provide continuous loading of the filtrate onto the at least two AEX columns for manufacturing the recombinant human lysosomal proteins.

In one or more embodiments, the filtrate is loaded on the at least two AEX columns at a filtrate load rate in the range of about 0.5 to about 100 column volumes (CV) per hour, such as about 1 to about 40 CV per hour.

In some embodiments, the filtrate is loaded on the at least two AEX columns to provide an AEX load time of less than 48 hours for each AEX column, such as less than 24 hours.

In some embodiments, each AEX column has a column volume of less than or equal to 50 L.

In one or more embodiments, the second biologic product is eluted from the one or more IMAC columns within 48 hours of removing the media and/or cell suspension from the bioreactor.

In one or more embodiments, the one or more IMAC columns have a total IMAC column volume and the ratio of the bioreactor volume to the total IMAC column volume is in the range of about 5,000:1 to about 50:1.

In some embodiments, ratio of the total AEX column volume to the total IMAC column volume is in the range of about 20:1 to about 1:1.

In some embodiments, each IMAC column has a column volume of less than or equal to 20 L.

In one or more embodiments, the method further comprises storing the second biologic product. In one or more embodiments, the second biologic product is stored at a temperature of 0° C. to 10° C. for a time period of 24 hours to 105 days. In one or more embodiments, the second biologic product is stored for up to 1, 2, 3, 4, 5, 10, 15, 20, 25, 30, 35, 40, 45, 50, 55, 60, 65, 70, 75, 80, 85, 90, 95, 100 or 105 days. In one or more embodiments, the second biologic product is stored at a temperature of 15° C. to 30° C. for a time period of 1 hour to 3 days.

In one or more embodiments, the method further comprises loading the second biologic product onto a third chromatography column; and eluting a third biologic product from the third chromatography column. In one or more embodiments, the third chromatography column is selected from an anion exchange chromatography (AEX) column, an affinity chromatography column, cation exchange chromatography (CEX) column, an immobilized metal affinity chromatography (IMAC) column, a size exclusion chromatography (SEC) column and a hydrophobic interaction chromatography (HIC) column.

In one or more embodiments, the filtrate is separated by filtering the media and/or cell suspension from one or more of alternating tangential flow filtration (ATF) and tangential flow filtration (TFF).

In one or more embodiments, the method further comprises inactivating viruses in one or more of the first biologic product, the second biologic product and the third biologic product.

In one or more embodiments, the method further comprises filtering the second biologic product or the third biologic product to provide a filtered product and filling a vial with the filtered product.

In one or more embodiments, the method further comprises lyophilizing the filtered product.

In one or more embodiments, the biologic comprises rhGAA. In one or more embodiments, the rhGAA comprises an amino acid sequence that is at least 95% identical to SEQ ID NO: 2.

In one or more embodiments, the host cells comprise Chinese hamster ovary (CHO) cells. In one or more embodiments, the host cells comprise CHO cell line GA-ATB-200 or ATB-200-001-X5-14 or a subculture thereof

In one or more embodiments, (i) at least 90% of the first biologic product or the second biologic product or the third biologic product binds to CIMPR and/or (ii) at least 90% of the first biologic product or the second biologic product or the third biologic product contains an N-glycan carrying mono-M6P or bis-M6P.

In one or more embodiments, the rhGAA comprises seven potential N-glycosylation sites, at least 50% of molecules of the rhGAA comprise an N-glycan unit bearing two mannose-6-phosphate residues at the first site, at least 30% of molecules of the rhGAA comprise an N-glycan unit bearing one mannose-6-phosphate residue at the second site, at least 30% of molecules of the rhGAA comprise an N-glycan unit bearing two mannose-6-phosphate residue at the fourth site, and at least 20% of molecules of the rhGAA comprise an N-glycan unit bearing one mannose-6-phosphate residue at the fourth site.

In one or more embodiments, 40%-60% of the N-glycans on the rhGAA are complex type N-glycans; and the rhGAA comprises 3.0-5.0 mol M6P residues per mol rhGAA.

Another aspect of the invention pertains to a method for manufacturing biologics. In various embodiments of this aspect, the method comprises culturing cells in a bioreactor and loading biologics-containing fluid onto at least two AEX columns, wherein the at least two AEX columns have a total AEX column volume, and wherein the ratio of the bioreactor volume to the total AEX column volume is in the range of about 500:1 to about 10:1, such as ratios of about 100:1 to about 20:1. In various embodiments of this aspect, the total AEX column residence time (i.e. the quotient of the total AEX column volume and the volumetric flow rate loading the AEX columns) is in the range of about 0.5 minutes to 200 minutes, such as about 10 to 70 minutes.

In one or more embodiments, the method comprises: culturing host cells in a bioreactor that secrete a recombinant human lysosomal protein; removing media from the bioreactor; filtering the media to provide a filtrate; loading the filtrate onto at least two AEX columns to capture the lysosomal protein; eluting a first protein product from the at least two AEX columns; loading the first protein product onto one or more IMAC columns; and eluting a second protein product from the one or more IMAC columns; wherein the bioreactor has a bioreactor volume, the at least two AEX columns have a total AEX column volume, and wherein the ratio of the bioreactor volume to the total AEX column volume is in the range of about 500:1 to about 10:1, such as ratios of about 100:1 to about 20:1.

In one or more embodiments, the at least two AEX columns are loaded sequentially to provide continuous loading of the filtrate onto the at least two AEX columns.

In one or more embodiments, the filtrate is loaded on the at least two AEX columns at a filtrate load rate in the range of about 0.5 to about 100 CV per hour, such as about 1 to about 40 CV per hour.

In one or more embodiments, the filtrate is loaded on the at least two AEX columns to provide an AEX load time of less than 48 hours for each AEX column, such as less than 24 hours.

In one or more embodiments, each AEX column has a column volume of less than or equal to 50 L.

In one or more embodiments, the second protein product is eluted from the one or more IMAC columns within 48 hours of removing the media from the bioreactor.

In one or more embodiments, the one or more IMAC columns have a total

IMAC column volume and the ratio of the bioreactor volume to the total IMAC column volume is in the range of about 5,000:1 to about 50:1.

In one or more embodiments, the ratio of the total AEX column volume to the total IMAC column volume is in the range of about 20:1 to about 1:1.

In one or more embodiments, each IMAC column has a column volume of less than or equal to 20 L.

In one or more embodiments, the method further comprises storing the second protein product. In one or more embodiments, the second protein product is stored at a temperature of 0° C. to 10° C. for a time period of 24 hours to 105 days. In one or more embodiments, the second protein product is stored for up to 1, 2, 3, 4, 5, 10, 15, 20, 25, 30, 35, 40, 45, 50, 55, 60, 65, 70, 75, 80, 85, 90, 95, 100 or 105 days. In one or more embodiments, the second protein product is stored at a temperature of 15° C. to 30° C. for a time period of 1 hour to 3 days.

In one or more embodiments, the method further comprises loading the second protein product onto a third chromatography column; and eluting a third protein product from the third chromatography column. In one or more embodiments, the third chromatography column is selected from a CEX column and an SEC column.

In one or more embodiments, filtering the media is selected from ATF and TFF.

In one or more embodiments, the method further comprises inactivating viruses in one or more of the first protein product, the second protein product and the third protein product.

In one or more embodiments, the method further comprises filtering the second protein product or the third protein product to provide a filtered product and filling a vial with the filtered product.

In one or more embodiments, the method further comprises lyophilizing the filtered product.

In one or more embodiments, the recombinant human lysosomal protein is rhGAA. In one or more embodiments, the rhGAA comprises an amino acid sequence that is at least 95% identical to SEQ ID NO: 2.

In one or more embodiments, the host cells comprise CHO cells. In one or more embodiments, the host cells comprise CHO cell line GA-ATB-200 or ATB-200-001-X5-14 or a subculture thereof

In one or more embodiments, (i) at least 90% of the first protein product or the second protein product or the third protein product binds CIMPR and/or (ii) at least 90% of the first protein product or the second protein product or the third protein product contains an N-glycan carrying mono-mannose-6-phosphate (mono-M6P) or bis-mannose-6-phosphate (bis-M6P).

In one or more embodiments, the rhGAA comprises seven potential N-glycosylation sites, at least 50% of molecules of the rhGAA comprise an N-glycan unit bearing two mannose-6-phosphate residues at the first site, at least 30% of molecules of the rhGAA comprise an N-glycan unit bearing one mannose-6-phosphate residue at the second site, at least 30% of molecules of the rhGAA comprise an N-glycan unit bearing two mannose-6-phosphate residue at the fourth site, and at least 20% of molecules of the rhGAA comprise an N-glycan unit bearing one mannose-6-phosphate residue at the fourth site.

In one or more embodiments, 40%-60% of the N-glycans on the rhGAA are complex type N-glycans; and the rhGAA comprises 3.0-5.0 mol M6P residues per mol rhGAA.

Another aspect of the present invention is related to a biologic product made by any of the methods described herein.

Another aspect of the present invention is related to pharmaceutical composition comprising the biologic product and a pharmaceutically acceptable carrier.

Yet another aspect of the present invention is related to a method for treating a lysosomal storage disorder, the method comprising administering the pharmaceutical composition to a patient in need thereof

In one or more embodiments, the lysosomal storage disorder is Pompe disease and the biologic product is rhGAA. In one or more embodiments, the patient is co-administered a pharmacological chaperone for α-glucosidase within 4 hours of the administration of the pharmaceutical composition comprising the rhGAA product. In some embodiments, the pharmacological chaperone is selected from 1-deoxynojirimycin and N-butyl-deoxynojirimycin. In some embodiments, the pharmacological chaperone is co-formulated with the rhGAA product.

Various embodiments are listed below. It will be understood that the embodiments listed below may be combined not only as listed below, but in other suitable combinations in accordance with the scope of the invention.

### BRIEF DESCRIPTION OF THE DRAWINGS

Further features of the present invention will become apparent from the following written description and the accompanying figures, in which:

FIG. 1A shows non-phosphorylated high mannose glycan, a mono-M6P glycan, and a bis-M6P glycan.

FIG. 1B shows the chemical structure of the M6P group.

FIG. 2A describes productive targeting of rhGAA via glycans bearing M6P to target tissues (e.g. muscle tissues of subject with Pompe Disease).

FIG. 2B describes non-productive drug clearance to non-target tissues (e.g.

liver and spleen) or by binding of non-M6P glycans to non-target tissues.

FIG. 3A graphically depicts a CIMPR receptor (also known as an IGF2 receptor) and domains of this receptor.

FIG. 3B is a table showing binding affinity (nmolar) of glycans bearing bis- and mono-M6P for CIMPR, the binding affinity of high mannose-type glycans to mannose receptors, and the binding affinity of desialylated complex glycan for asialyoglycoprotein receptors. RhGAA that has glycans bearing M6P and bis-M6P can productively bind to CIMPR on muscle.

FIG. 4 shows a DNA construct for transforming CHO cells with DNA encoding rhGAA. CHO cells were transformed with a DNA construct encoding rhGAA.

FIG. 5 is a schematic diagram of an exemplary prior art process for the manufacturing, capturing and purification of a recombinant protein.

FIG. 6 is a schematic diagram of an exemplary process for the manufacturing, capturing and purification of biologics according to one or more embodiments of the invention.

FIG. 7 describes exemplary first sequence of events in manufacturing, capturing and purification of biologics, wherein the filtrate containing biologics is loaded on the capture column 1.

FIG. 8 describes exemplary second sequence of events in manufacturing, capturing and purification of biologics, wherein the captured biologics in the capture column 1 is eluted and loaded onto the purification column. During the process, the filtrate containing biologics is loaded on the capture column 2.

FIG. 9 describes exemplary third sequence of events in manufacturing, capturing and purification of biologics, wherein the captured biologics in the capture column 2 is eluted and loaded onto the purification column. During the process, the filtrate containing biologics is loaded on the capture column 1.

FIG. 10 describes exemplary fourth sequence of events in manufacturing, capturing and purification of biologics, wherein the biologic is eluted from the purification column.

FIG. 11 describes exemplary sequence of events in manufacturing, capturing and purification of rhGAA, wherein AEX columns are used as capture column and IMAC column is used as purification column.

FIG. 12 is a schematic diagram of another exemplary process for the manufacturing, capturing and purification of biologics according to one or more embodiments of the invention.

FIG. 13 shows the elution profile from two AEX capture columns in a batch purification process.

FIG. 14 shows the elution profile from two AEX capture columns in a continuous purification process.

FIG. 15 shows a comparison of elution profile from an IMAC purification column between a batch purification process and a continuous purification process.

FIG. 16 shows an enlarged image of the elution profile from an IMAC purification column between a batch purification process and a continuous purification process from FIG. 15.

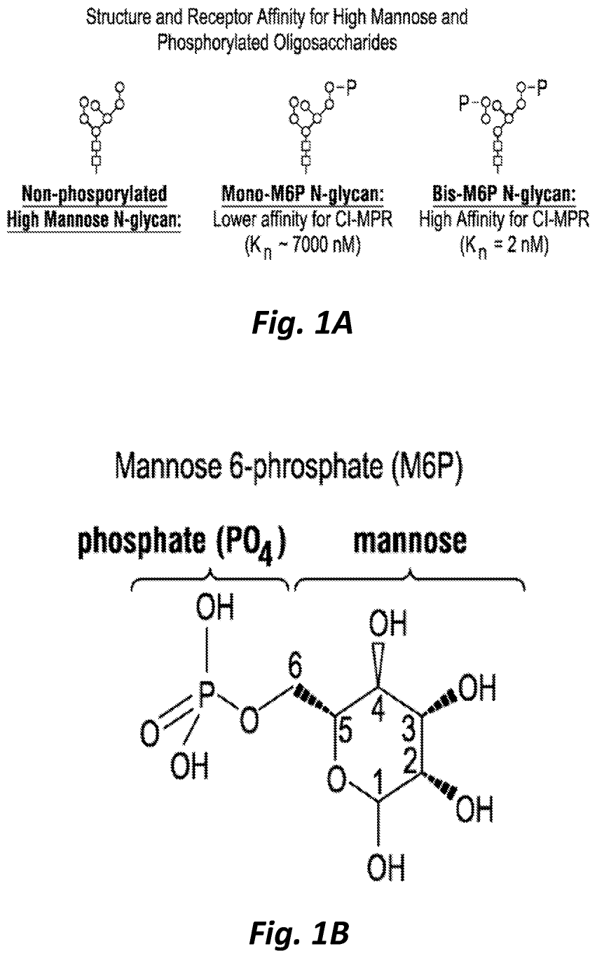

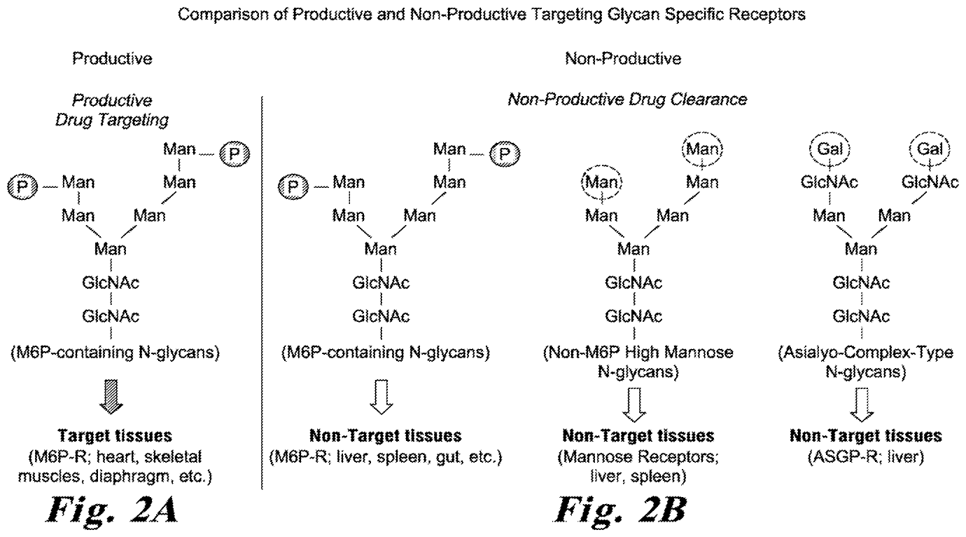

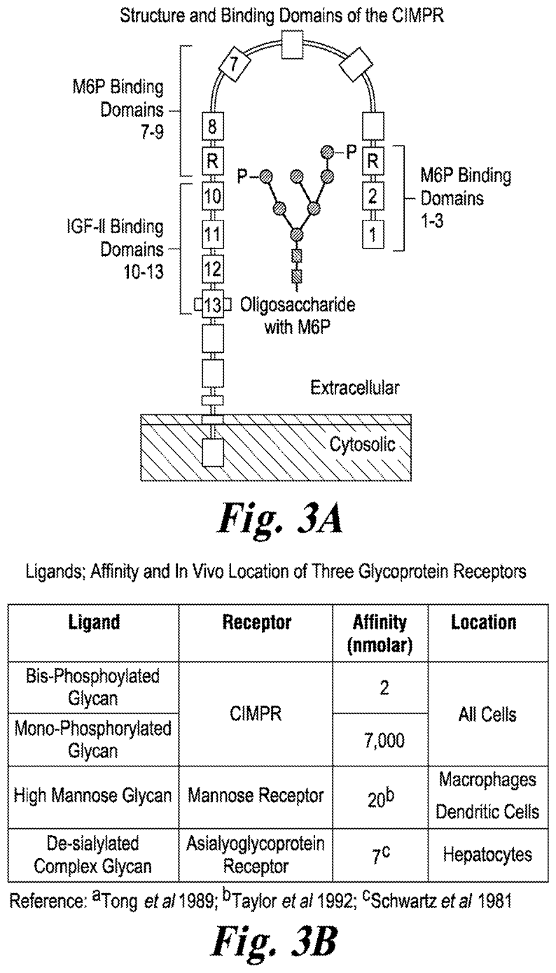

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

The disclosure describes the method of producing, capturing and purifying biologics. In one or more embodiments, the biologics comprise one or more of a recombinant protein, a virus particle or an antibody. In one or more embodiments, the recombinant proteins target lysosomes. In one or more embodiments, the recombinant protein is recombinant human α-galactosidase A (rhGAA).

In one or more embodiments, the recombinant proteins undergo post-translation and/or chemical modifications at one or more amino acid residues in the protein. For example, methionine and tryptophan residues can undergo oxidation. As another example, the N-terminal glutamine can form pyro-glutamate. As another example, asparagine residues can undergo deamidation to aspartic acid. As yet another example, aspartic acid residues can undergo isomerization to iso-aspartic acid. As yet another example, unpaired cysteine residues in the protein can form disulfide bonds with free glutathione and/or cysteine.

Although specific reference is made to rhGAA, it will be understood by a person having ordinary skill in the art that the methods and systems described herein may be used to produce, capture and purify other recombinant proteins. In various embodiments, the other recombinant proteins also target the lysosome, including but not limited to the lysosomal enzyme α-galactosidase A. Also, the methods and systems described herein can also be used to produce, capture and purify other biologic products such as antibodies and virus particles (e.g. for gene therapy).

Some current methods for the production of rhGAA utilize large AEX columns, such as AEX columns with dimensions of 1 meter diameter by 30 cm bed height (i.e. each AEX column volume =236 L). These large AEX columns have long loading times (e.g. 96 hours), and due to the low stability of rhGAA during AEX conditions, the AEX is performed in cold rooms with a controlled temperature of 2° C.-8° C. The eluate from the large AEX columns are then manually loaded on large IMAC columns, such as IMAC columns with dimensions of 60 cm diameter by 20 cm bed height (i.e. each IMAC column volume =56.5 L). These manufacturing systems with large AEX and IMAC columns also have several disadvantages: a large facility footprint; poor productivity (enzyme produced per liter of resin per hour); high operator involvement (increases risk of human error); and high product loss/rejection if something goes wrong with an AEX cycle or forward processing on IMAC is delayed.

However, it has surprisingly been discovered that a relatively compact manufacturing system can provide one or more of the following advantages: reduce capture column (e.g. AEX) load times; eliminate cold room processing; reduce facility footprint; improve productivity; reduce operator involvement due to straight through processing from capture column (e.g. AEX) to purification column (e.g. IMAC); and minimize product loss/rejection if something goes wrong with a biologic capture (e.g. AEX) cycle. Additionally, the system uses a smaller column size, which helps achieving better separation of end product from other proteins.

Accordingly, various aspects of the invention pertain to new methods for the production, capturing and purification of biologics (e.g. recombinant proteins, including recombinant human lysosomal proteins such as rhGAA). Other aspects of the invention pertain to biologics (e.g. recombinant proteins) produced by the processes described herein, as well as pharmaceutical compositions, methods of treatment, and uses of such biologics (e.g. recombinant proteins).

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

In the present specification, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

As used herein, the term “bioreactor volume” refers to the working volume (i.e. liquid volume) within the bioreactor. The working volume within the bioreactor may be less than 10000 liters, less than 5000 liters, less than 4000 liters, less than 3500 liters, less than 3000 liters, less than 2500 liters, less than 2000 liters, less than 1500 liters, less than 1000 liters, less than 500 liters or less than 250 liters.

As used herein, the term “capture column” refers to a chromatography column that captures a desired biological product produced from a bioreactor. The disclosure describes a method to use at least two capture columns. In various embodiments, the at least two capture columns are loaded sequentially to provide continuous loading of a filtrate onto the at least two capture columns.

As used herein, the term “purification column” refers to a chromatography column that is used to further purify a desired biological product after it has been captured on a capture column.

As used herein, the term “column volume” refers to the packed bed volume of a chromatography column.

As used herein, the term “total . . . column volume” such as “total biologics capture column volume”, total AEX column volume and the like refers to the aggregate column volume of all columns of the specified type.

As used herein, the term “total . . . column residence time” refers to the quotient of the aggregate column volume of all columns of the specified type and the volumetric flow rate used to load the columns.

As used herein, the term “recombinant DNA” refers to DNA that has been formed artificially by combining genetic material from multiple sources (e.g. different organisms).

As used herein, the term “biologics” or “biologic” or “biologics product” refers to a complex molecule or mixture of molecules produced in a living system. Biologics are often produced in cell-based systems using recombinant DNA technology. Examples of biologics include, but are not limited to, a recombinant protein, a virus particle and an antibody.

As used herein, the term “recombinant protein” refers to a protein encoded by a gene in recombinant DNA that has been cloned in a system that supports expression of the gene. In one or more embodiments, the recombinant protein is a secreted protein or an intracellular protein that is produced in a host cell. The host cell may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

In some embodiments, the recombinant protein may be a secreted protein, a membrane protein or an intracellular protein. The secreted protein may be separated either by a filtration or a centrifugation into the filtrate. The intracellular protein may be separated by first lysing the cells followed by either filtration or centrifugation into the filtrate. The membrane proteins may be separated by lysing the cells, separating the recombinant containing membrane by ultracentrifugation and solubilizing the membrane protein using a suitable detergent and preparing the filtrate by ultracentrifugation to separate non-soluble membrane proteins. The detergent may be anionic, cationic or zwitterionic in nature.

As used herein, the term “lysosomal protein” refers to any protein that is targeted to the lysosome, such as a lysosomal enzyme. Examples of lysosomal enzymes and the associated disease include, but are not limited to, those provided in Table 1 below:

TABLE 1

| Lysosomal Enzyme | Disease |
|---|---|
| Acid α-glucosidase | Pompe disease |
| α-galactosidase A | Fabry disease |
| Acid β-glucosidase | Gaucher disease |
| α-L-iduronidase | Hurler-Scheie disease |
| Iduronate sulfatase | Hunter disease |
| β-galactosidase | GM1-gangliosidosis; Morquio |
| | disease B |
| β-glucuronidase | Sly disease (MPS VI) |
| α-fucosidase | Fucosidosis |
| Acid sphingomyelinase | Niemann-Pick A and B |
| β-hexosaminidase A | Tay Sachs disease |
| β-hexosaminidase B | Sandhoff disease |
| β-galactocerebrosidase | Krabbe disease |
| Acid ceramidase | Farber disease |
| Heparan-N-sulfatase | Sanfilippo disease A (MPS IIIa) |
| α-N-acetyl-glucosaminidase | Sanfilippo disease B (MPS IIIb) |
| α-glucosaminide N-acetyltransferase | Sanfilippo disease C (MPS IIIc) |
| N-acetylglucosamine-6-sulfate | Sanfilippo disease D (MPS IIId) |
| sulfatase | |
| N-acetylgalactosamine-6-sulfate | Morquio disease A (MPS Ivb) |
| sulfatase | |
| Arylsulfatase A | Metachromatic leukodystrophy |
| Arylsulfatase B | Maroteaux-Lamy (MPS VI) |
| Acid lipase | Wolf disease |
| acid α-mannosidase | α-mannosidosis |
| acid β-mannosidase | β-mannosidosis |
| α-N-acetyl-neuraminidase | Sialidosis |
| α-N-acetylgalactosaminidase | Schindler-Kanzaki disease |
| N-aspartyl-β-glucosaminidase | Aspartylglucosaminuria |

In one or more embodiments, the lysosomal protein is selected from the group consisting of alpha-galactosidase (A or B), β-galactosidase, β-hexosaminidase (A or B), galactosylceramidase, arylsulfatase (A or B), β-glucocerebrosidase, glucocerebrosidase, lysosomal acid lipase, lysosomal enzyme acid sphingomyelinase, formylglycine-generating enzyme, iduronidase (e.g., alpha-L), acetyl-CoA: alpha-glucosaminide N-acetyltransferase, glycosaminoglycan alpha-L-iduronohydrolase, heparan N-sulfatase, N-acetyl-alpha-D-glucosaminidase, iduronate-2-sulfatase, galactosamine-6-sulfate sulfatase, N-acetylgalactosamine-6-sulfatase, glycosaminoglycan N-acetylgalactosamine 4-sulfatase, (β-glucuronidase, hyaluronidase, alpha-N -acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins (e.g., ceroid-lipofuscinosis neuronal protein 6). In some embodiments, the therapeutic protein is an alpha-galactosidase. In some embodiments, the enzyme is a palmitoyl protein thioesterase (PPT)—including palmitoyl protein thioesterase 1 and 2 (PPT1 and PPT2 respectively). In some embodiments, the enzyme is palmitoyl protein thioesterase 1. In some embodiments, the therapeutic protein is associated with a genetic disorder selected from the group consisting of CDKL5 deficiency disorder, cystic fibrosis, alpha- and beta-thalassemias, sickle cell anemia, Marfan syndrome, fragile X syndrome, Huntington's disease, hemochromatosis, Congenital Deafness (nonsyndromic), Tay-Sachs, Familial hypercholesterolemia, Duchenne muscular dystrophy, Stargardt disease, Usher syndrome, choroideremia, achromatopsia, X-linked retinoschisis, hemophilia, Wiskott-Aldrich syndrome, X-linked chronic granulomatous disease, aromatic L-amino acid decarboxylase deficiency, recessive dystrophic epidermolysis bullosa, alpha 1 antitrypsin deficiency, Hutchinson-Gilford progeria syndrome (HGPS), Noonan syndrome, X-linked severe combined immunodeficiency (X-SCID). In some embodiments, the therapeutic protein is selected from the group consisting of CDKL5, Connexin 26, hexosaminidase A, LDL receptor, Dystrophin, CFTR, beta-globulin, HFE, Huntington, ABCA4, myosin VIIA (MYO7A), Rab escort protein-1 (REP1), cyclic nucleotide gated channel beta 3 (CNGB3), retinoschisin 1 (RS1), hemoglobin subunit beta (HBB), Factor IX, WAS, cytochrome B-245 beta chain, dopa decarboxylase (DDC), collagen type VII alpha 1 chain (COL7A1), serpin family A member 1 (SERPINA1), LMNA, PTPN11, SOS1, RAF1, KRAS, and IL2 receptor y gene.

In one or more embodiments, the genetic disorder (e.g. lysosomal storage disorder) is selected from the group consisting of aspartylglucosaminuria, Batten disease, cystinosis, Fabry disease, Gaucher disease type I, Gaucher disease type II, Gaucher disease type III, Pompe disease, Tay Sachs disease, Sandhoff disease, metachromatic leukodystrophy, mucolipidosis type I, mucolipidosis type II, mucolipidosis type III, mucolipidosis type IV, Hurler disease, Hunter disease, Sanfilippo disease type A, Sanfilippo disease type B, Sanfilippo disease type C, Sanfilippo disease type D, Morquio disease type A, Morquio disease type B, Maroteau-Lamy disease, Sly disease, Niemann-Pick disease type A, Niemann-Pick disease type B, Niemann-Pick disease type Cl, Niemann-Pick disease type C2, Schindler disease type I, and Schindler disease type II. In some embodiments, the lysosomal storage disorder is selected from the group consisting of activator deficiency, GM2-gangliosidosis; GM2-gangliosidosis, AB variant; alpha-mannosidosis (type 2, moderate form; type 3, neonatal, severe); beta-mannosidosis; lysosomal acid lipase deficiency; cystinosis (late-onset juvenile or adolescent nephropathic type; infantile nephropathic); Chanarin-Dorfman syndrome; neutral lipid storage disease with myopathy; NLSDM; Danon disease; Fabry disease; Fabry disease type II, late-onset; Farber disease; Farber lipogranulomatosis; fucosidosis; galactosialidosis (combined neuraminidase & beta-galactosidase deficiency); Gaucher disease ; type II Gaucher disease ; type III Gaucher disease; type IIIC Gaucher disease; Gaucher disease, atypical, due to saposin C deficiency; GM1-gangliosidosis (late-infantile/juvenile GM1-gangliosidosis; adult/chronic GM1-gangliosidosis); Globoid cell leukodystrophy, Krabbe disease (Late infantile onset; Juvenile Onset; Adult Onset); Krabbe disease, atypical, due to saposin A deficiency; Metachromatic Leukodystrophy (juvenile; adult); partial cerebroside sulfate deficiency; pseudoarylsulfatase A deficiency; metachromatic leukodystrophy due to saposin B deficiency; Mucopolysaccharidoses disorders: MPS I, Hurler syndrome; MPS I, Hurler-Scheie syndrome; MPS I, Scheie syndrome ; MPS II, Hunter syndrome ; MPS II, Hunter syndrome ; Sanfilippo syndrome Type A/MPS IIIA ; Sanfilippo syndrome Type B/MPS IIIB ; Sanfilippo syndrome Type C/MPS IIIC ; Sanfilippo syndrome Type D/MPS IIID ; Morquio syndrome, type A/MPS IVA ; Morquio syndrome, type B/MPS IVB ; MPS IX hyaluronidase deficiency ; MPS VI Maroteaux-Lamy syndrome ; MPS VII Sly syndrome; mucolipidosis I, sialidosis type II; I-cell disease, Leroy disease, mucolipidosis II; Pseudo-Hurler polydystrophy/mucolipidosis type III; mucolipidosis IIIC/ML III GAMMA; mucolipidosis type IV; multiple sulfatase deficiency; Niemann-Pick disease (type B; type Cl/chronic neuronopathic form; type C2; type D/Nova Scotian type); Neuronal Ceroid Lipofuscinoses: CLN6 disease-Atypical Late Infantile, Late-Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/Adult-onset NCL/CLN4 disease (type B); Northern Epilepsy/variant late infantile CLN8; Santavuori-Haltia/Infantile CLN1/PPT disease; Pompe disease (glycogen storage disease type II); late-onset Pompe disease; Pycnodysostosis; Sandhoff disease/GM2 gangliosidosis; Sandhoff disease/GM2 gangliosidosis; Sandhoff disease/GM2 Gangliosidosis; Schindler disease (type III/intermediate, variable); Kanzaki disease; Salla disease; infantile free sialic acid storage disease (ISSD); spinal muscular atrophy with progressive myoclonic epilepsy (SMAPME); Tay-Sachs disease/GM2 gangliosidosis; juvenile-onset Tay-Sachs disease; late-onset Tay-Sachs disease; Christianson syndrome; Lowe oculocerebrorenal syndrome; Charcot-Marie-Tooth type 4J, CMT4J; Yunis-Varon syndrome; bilateral temporooccipital polymicrogyria (BTOP); X-linked hypercalciuric nephrolithiasis, Dent-1; and Dent disease 2, adenosine deaminase severe combined immunodeficiency (ADA-SCID), and neuronal ceroid lipofuscinosis.

As used herein, the term “Pompe disease,” also referred to as acid maltase deficiency, glycogen storage disease type II (GSDII), and glycogenosis type II, is intended to refer to a genetic lysosomal storage disorder characterized by mutations in the GAA gene, which codes for the human acid α-glucosidase enzyme. The term includes but is not limited to early and late onset forms of the disease, including but not limited to infantile, juvenile and adult-onset Pompe disease.

As used herein, the term “acid α-glucosidase” is intended to refer to a lysosomal enzyme which hydrolyzes α-1,4 linkages between the D-glucose units of glycogen, maltose, and isomaltose. Alternative names include but are not limited to lysosomal α-glucosidase (EC:3.2.1.20); glucoamylase; 1,4-α-D-glucan glucohydrolase; amyloglucosidase; gamma-amylase and exo-1,4-α-glucosidase. Human acid α-glucosidase is encoded by the GAA gene (National Centre for Biotechnology Information (NCBI) Gene ID 2548), which has been mapped to the long arm of chromosome 17 (location 17q25.2-q25.3). More than 500 mutations have currently been identified in the human GAA gene, many of which are associated with Pompe disease. Mutations resulting in misfolding or misprocessing of the acid α-glucosidase enzyme include T1064C (Leu355Pro) and C2104T (Arg702Cys). In addition, GAA mutations which affect maturation and processing of the enzyme include Leu405Pro and Met519Thr. The conserved hexapeptide WIDMNE at amino acid residues 516-521 is required for activity of the acid α-glucosidase protein. As used herein, the abbreviation “GAA” is intended to refer to the acid α-glucosidase enzyme, while the italicized abbreviation “GAA” is intended to refer to the human gene coding for the human acid α-glucosidase enzyme The italicized abbreviation “Gaa” is intended to refer to non-human genes coding for non-human acid α-glucosidase enzymes, including but not limited to rat or mouse genes, and the abbreviation “Gaa” is intended to refer to non-human acid α-glucosidase enzymes. Thus, the abbreviation “rhGAA” is intended to refer to the recombinant human acid α-glucosidase enzyme.

As used herein, the term “alglucosidase alfa” is intended to refer to a recombinant human acid α-glucosidase identified as [199-arginine,223-histidine]prepro-α-glucosidase (human); Chemical Abstracts Registry Number 420794-05-0. Alglucosidase alfa is approved for marketing in the United States by Genzyme, as of January 2016, as the products Lumizyme® and Myozyme®.

As used herein, the term “ATB200” is intended to refer to a recombinant human acid α-glucosidase described in PCT patent application PCT/US2015/053252, now issued as U.S. Pat. No. 10,208,299, the disclosure of which is herein incorporated by reference in its entirety. Methods of manufacturing recombinant lysosomal proteins (including rhGAA such as ATB200) are described in U.S. Pat. No. 10,227,577, which is also incorporated by reference in its entirety. Formulations and methods using rhGAA are described in co-pending Application Publication nos. US 2017/0333534 and US 2018/0228877, which are also incorporated by reference in their entirety.

As used herein, the term “glycan” is intended to refer to a polysaccharide chain covalently bound to an amino acid residue on a protein or polypeptide. As used herein, the term “N-glycan” or “N-linked glycan” is intended to refer to a polysaccharide chain attached to an amino acid residue on a protein or polypeptide through covalent binding to a nitrogen atom of the amino acid residue. For example, an N-glycan can be covalently bound to the side chain nitrogen atom of an asparagine residue. Glycans can contain one or several monosaccharide units, and the monosaccharide units can be covalently linked to form a straight chain or a branched chain. In at least one embodiment, N-glycan units attached to ATB200 can comprise one or more monosaccharide units each independently selected from N-acetylglucosamine, mannose, galactose or sialic acid. The N-glycan units on the protein can be determined by any appropriate analytical technique, such as mass spectrometry. In some embodiments, the N-glycan units can be determined by liquid chromatography-tandem mass spectrometry (LC-MS/MS) utilizing an instrument such as the Thermo Scientific Orbitrap Velos Pro™ Mass Spectrometer, Thermo Scientific Orbitrap Fusion Lumos Tribid™ Mass Spectrometer or Waters Xevo® G2-XS QTof Mass Spectrometer.

As used herein, the term “high-mannose N-glycan” is intended to refer to an N-glycan having one to six or more mannose units. In at least one embodiment, a high mannose N-glycan unit can contain a bis(N-acetylglucosamine) chain bonded to an asparagine residue and further bonded to a branched polymannose chain. As used herein interchangeably, the term “M6P” or “mannose-6-phosphate” is intended to refer to a mannose unit phosphorylated at the 6 position; i.e. having a phosphate group bonded to the hydroxyl group at the 6 position. In at least one embodiment, one or more mannose units of one or more N-glycan units are phosphorylated at the 6 position to form mannose-6-phosphate units. In at least one embodiment, the term “M6P” or “mannose-6-phosphate” refers to both a mannose phosphodiester having N-acetylglucosamine (GlcNAc) as a “cap” on the phosphate group, as well as a mannose unit having an exposed phosphate group lacking the GlcNAc cap. In at least one embodiment, the N-glycans of a protein can have multiple M6P groups, with at least one

M6P group having a GlcNAc cap and at least one other M6P group lacking a GlcNAc cap.

As used herein, the term “complex N-glycan” is intended to refer to an N-glycan containing one or more galactose and/or sialic acid units. In at least one embodiment, a complex N-glycan can be a high-mannose N-glycan in which one or mannose units are further bonded to one or more monosaccharide units each independently selected from N-acetylglucosamine, galactose and sialic acid.

As used herein, the compound miglustat, also known as N-butyl-1-deoxynojirimycin NB-DNJ or (2R,3R,4R,5 S)-1-butyl-2-(hydroxymethyl)piperidine-3,4,5 -triol, is a compound having the following chemical formula:

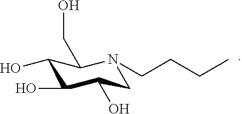

One formulation of miglustat is marketed commercially under the trade name

Zavesca® as monotherapy for type 1 Gaucher disease.

As discussed below, pharmaceutically acceptable salts of miglustat may also be used in the present invention. When a salt of miglustat is used, the dosage of the salt will be adjusted so that the dose of miglustat received by the patient is equivalent to the amount which would have been received had the miglustat free base been used.

As used herein, the compound duvoglustat, also known as 1-deoxynojirimycin or DNJ or (2R,3R,4R,5S)-2-(hydroxymethyl)piperidine-3,4,5-triol, is a compound having the following chemical formula:

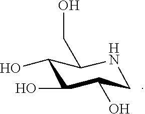

When a salt of duvoglustat is used, the dosage of the salt will be adjusted so that the dose of duvoglustat received by the patient is equivalent to the amount which would have been received had the duvoglustat free base been used.

As used herein, the term “pharmacological chaperone” or sometimes simply the term “chaperone” is intended to refer to a molecule that specifically binds to a protein (e.g. naturally occurring proteins or recombinant proteins) and has one or more of the following effects:
enhances the formation of a stable molecular conformation of the protein;
enhances proper trafficking of the protein from the endoplasmic reticulum to another cellular location, preferably a native cellular location, so as to prevent endoplasmic reticulum-associated degradation of the protein;
prevents aggregation of conformationally unstable or misfolded proteins;
restores and/or enhances at least partial wild-type function, stability, and/or activity of the protein; and/or
improves the phenotype or function of the cell harboring the protein.

Thus, a pharmacological chaperone includes lysosomal protein. For example, a chaperone for acid α-glucosidase is a molecule that binds to acid α-glucosidase, resulting in proper folding, trafficking, non-aggregation, and activity of acid α-glucosidase. As used herein, this term includes but is not limited to active site-specific chaperones (ASSCs) which bind in the active site of the enzyme, inhibitors or antagonists, and agonists. In at least one embodiment, the pharmacological chaperone can be an inhibitor or antagonist of acid α-glucosidase. As used herein, the term “antagonist” is intended to refer to any molecule that binds to acid α-glucosidase and either partially or completely blocks, inhibits, reduces, or neutralizes an activity of acid α-glucosidase. In at least one embodiment, the pharmacological chaperone is miglustat. Another non-limiting example of a pharmacological chaperone for acid α-glucosidase is duvoglustat.

As used herein, the term “active site” is intended to refer to a region of a protein that is associated with and necessary for a specific biological activity of the protein. In at least one embodiment, the active site can be a site that binds a substrate or other binding partner and contributes the amino acid residues that directly participate in the making and breaking of chemical bonds.

As used herein, the “therapeutically effective dose” and “effective amount” are intended to refer to an amount of recombinant protein (e.g. rhGAA) and/or of chaperone and/or of a combination thereof, which is sufficient to result in a therapeutic response in a subject. A therapeutic response may be any response that a user (for example, a clinician) will recognize as an effective response to the therapy, including any surrogate clinical markers or symptoms described herein and known in the art. Thus, in at least one embodiment, a therapeutic response can be an amelioration or inhibition of one or more symptoms or markers of Pompe disease such as those known in the art. Symptoms or markers of Pompe disease include but are not limited to decreased acid α-glucosidase tissue activity; cardiomyopathy; cardiomegaly; progressive muscle weakness, especially in the trunk or lower limbs; profound hypotonia; macroglossia (and in some cases, protrusion of the tongue); difficulty swallowing, sucking, and/or feeding; respiratory insufficiency; hepatomegaly (moderate); laxity of facial muscles; areflexia; exercise intolerance; exertional dyspnea; orthopnea; sleep apnea; morning headaches; somnolence; lordosis and/or scoliosis; decreased deep tendon reflexes; lower back pain; and failure to meet developmental motor milestones. It should be noted that a concentration of chaperone (e.g. miglustat) that has an inhibitory effect on acid α-glucosidase may constitute an “effective amount” for purposes of this invention because of dilution (and consequent shift in binding due to the change in equilibrium), bioavailability and metabolism of the chaperone upon administration in vivo.

As used herein, the term “enzyme replacement therapy” or “ERT” is intended to refer to the introduction of a non-native, purified enzyme into an individual having a deficiency in such enzyme. The administered protein can be obtained from natural sources or by recombinant expression. The term also refers to the introduction of a purified enzyme in an individual otherwise requiring or benefiting from administration of a purified enzyme. In at least one embodiment, such an individual suffers from enzyme insufficiency. The introduced enzyme may be a purified, recombinant enzyme produced in vitro, or a protein purified from isolated tissue or fluid, such as, for example, placenta or animal milk, or from plants.

As used herein, the term “combination therapy” is intended to refer to any therapy wherein two or more individual therapies are administered concurrently or consecutively. In at least one embodiment, the results of the combination therapy are enhanced as compared to the effect of each therapy when it is performed individually. Enhancement may include any improvement of the effect of the various therapies that may result in an advantageous result as compared to the results achieved by the therapies when performed alone. Enhanced effect or results can include a synergistic enhancement, wherein the enhanced effect is more than the additive effects of each therapy when performed by itself; an additive enhancement, wherein the enhanced effect is substantially equal to the additive effect of each therapy when performed by itself; or less than a synergistic effect, wherein the enhanced effect is lower than the additive effect of each therapy when performed by itself, but still better than the effect of each therapy when performed by itself. Enhanced effect may be measured by any means known in the art by which treatment efficacy or outcome can be measured.

As used herein, the term “virus particle” is intended to include genetic material (e.g. DNA or RNA) surrounded by a protein coat known as a capsid. Examples of virus particles include, but are not limited to, an adeno-associated virus (AAV), a retrovirus, a lentivirus, a herpes simplex virus and an adenovirus. In one or more embodiments, the virus particle includes the recombinant DNA encoding a recombinant protein. In one or more embodiments, the virus particle includes additional elements for increasing expression and/or stabilizing the vector such as promoters (e.g., hybrid CBA promoter (CBh) and human synapsin 1 promoter (hSyn1)), a polyadenylation signals (e.g. Bovine growth hormone polyadenylation signal (bGHpolyA)), stabilizing elements (e.g. Woodchuck Hepatitis Virus (WHP) Posttranscriptional Regulatory Element (WPRE)) and/or an SV40 intron. In one or more embodiments, a vector may comprise a polynucleotide sequence flanking by regions that promote homologous recombination at a desired site in the genome, thus providing for expression of the desired protein (See Koller and Smithies, 1989, Proc. Natl. Acad. Sci. USA, 86:8932-8935; Zijlstra et al., 1989, Nature 342:435-438; U.S. Pat. No. 6,244,113 to Zarling et al.; and U.S. Pat. No. 6,200,812 to Pati et al.).

As used herein, the term “antibody” refers to an immunoglobulin, wherein the immunoglobulin includes natural and/or recombinant immunoglobulins. The source of natural immunoglobulin may be a mammal, including humans, domestic and farm animals, and laboratory, zoo, sports, or pet animals, such as dogs, horses, cats, cows, sheep, goats, pigs, mice, rats, rabbits, guinea pigs, monkeys. The source may be naturally or artificially exposed to a specific antigen to induce immunogenic response resulting in antibody production. Alternatively, the recombinant immunoglobulins may also be produces in the suitable host cells.

As used herein, the term “pharmaceutically acceptable” is intended to refer to molecular entities and compositions that are physiologically tolerable and do not typically produce untoward reactions when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans.

As used herein, the term “carrier” is intended to refer to a diluent, adjuvant, excipient, or vehicle with which a compound is administered. Suitable pharmaceutical carriers are known in the art and, in at least one embodiment, are described in “Remington's Pharmaceutical Sciences” by E. W. Martin, 18th Edition, or other editions.

As used herein, the terms “subject” or “patient” are intended to refer to a human or non-human animal. In at least one embodiment, the subject is a mammal. In at least one embodiment, the subject is a human.

As used herein, the term “anti-drug antibody” is intended to refer to an antibody specifically binding to a drug administered to a subject and generated by the subject as at least part of a humoral immune response to administration of the drug to the subject. In at least one embodiment the drug is a therapeutic protein drug product. The presence of the anti-drug antibody in the subject can cause immune responses ranging from mild to severe, including but not limited to life-threatening immune responses which include but are not limited to anaphylaxis, cytokine release syndrome and cross-reactive neutralization of endogenous proteins mediating critical functions. In addition or alternatively, the presence of the anti-drug antibody in the subject can decrease the efficacy of the drug.

As used herein, the term “neutralizing antibody” is intended to refer to an anti-drug antibody acting to neutralize the function of the drug. In at least one embodiment, the therapeutic protein drug product is a counterpart of an endogenous protein for which expression is reduced or absent in the subject. In at least one embodiment, the neutralizing antibody can act to neutralize the function of the endogenous protein.

As used herein, the terms “about” and “approximately” are intended to refer to an acceptable degree of error for the quantity measured given the nature or precision of the measurements. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “concurrently” as used herein is intended to mean at the same time as or within a reasonably short period of time before or after, as will be understood by those skilled in the art. For example, if two treatments are administered concurrently with each other, one treatment can be administered before or after the other treatment, to allow for time needed to prepare for the later of the two treatments. Therefore “concurrent administration” of two treatments includes but is not limited to one treatment following the other by 20 minutes or less, about 20 minutes, about 15 minutes, about 10 minutes, about 5 minutes, about 2 minutes, about 1 minute or less than 1 minute.

The term “pharmaceutically acceptable salt” as used herein is intended to mean a salt which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 66, pp. 1-19, herein incorporated by reference.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like.

ATB200 rhGAA

In at least one embodiment, the recombinant protein (e.g. recombinant protein such as rhGAA) is expressed in Chinese hamster ovary (CHO) cells and comprises an increased content of N-glycan units bearing one or more mannose-6-phosphate residues when compared to a content of N-glycan units bearing one or more mannose-6-phosphate residues of a conventional recombinant protein such as alglucosidase alfa. In at least one embodiment, the acid α-glucosidase is a recombinant human acid α-glucosidase referred to herein as ATB200, as described in U.S. Pat. No. 10,208,299. ATB200 has been shown to bind cation-independent mannose-6-phosphate receptors (CIMPR) with high affinity (K_{D}˜2-4 nM) and to be efficiently internalized by Pompe fibroblasts and skeletal muscle myoblasts (Kᵤₚₜₐₖₑ˜7-14 nM). ATB200 was characterized in vivo and shown to have a shorter apparent plasma half-life (t_{1/2}˜45 min) than alglucosidase alfa (t_{b 1/2}˜60 min).

In at least one embodiment, the recombinant human acid α-glucosidase is an enzyme having an amino acid sequence as set forth in SEQ ID NO: 1 or SEQ ID NO: 2

SEQ ID NO: 1
Met Gly Val Arg His Pro Pro Cys Ser His Arg Leu Leu Ala Val Cys
Ala Leu Val Ser Leu Ala Thr Ala Ala Leu Leu Gly His Ile Leu Leu
His Asp Phe Leu Leu Val Pro Arg Glu Leu Ser Gly Ser Ser Pro Val
Leu Glu Glu Thr His Pro Ala His Gln Gln Gly Ala Ser Arg Pro Gly
Pro Arg Asp Ala Gln Ala His Pro Gly Arg Pro Arg Ala Val Pro Thr
Gln Cys Asp Val Pro Pro Asn Ser Arg Phe Asp Cys Ala Pro Asp Lys
Ala Ile Thr Gln Glu Gln Cys Glu Ala Arg Gly Cys Cys Tyr Ile Pro
Ala Lys Gln Gly Leu Gln Gly Ala Gln Met Gly Gln Pro Trp Cys Phe
Phe Pro Pro Ser Tyr Pro Ser Tyr Lys Leu Glu Asn Leu Ser Ser Ser
Glu Met Gly Tyr Thr Ala Thr Leu Thr Arg Thr Thr Pro Thr Phe Phe
Pro Lys Asp Ile Leu Thr Leu Arg Leu Asp Val Met Met Glu Thr Glu
Asn Arg Leu His Phe Thr Ile Lys Asp Pro Ala Asn Arg Arg Tyr Glu
Val Pro Leu Glu Thr Pro Arg Val His Ser Arg Ala Pro Ser Pro Leu
Tyr Ser Val Glu Phe Ser Glu Glu Pro Phe Gly Val Ile Val His Arg
Gln Leu Asp Gly Arg Val Leu Leu Asn Thr Thr Val Ala Pro Leu Phe
Phe Ala Asp Gln Phe Leu Gln Leu Ser Thr Ser Leu Pro Ser Gln Tyr
Ile Thr Gly Leu Ala Glu His Leu Ser Pro Leu Met Leu Ser Thr Ser
Trp Thr Arg Ile Thr Leu Trp Asn Arg Asp Leu Ala Pro Thr Pro Gly
Ala Asn Leu Tyr Gly Ser His Pro Phe Tyr Leu Ala Leu Glu Asp Gly
Gly Ser Ala His Gly Val Phe Leu Leu Asn Ser Asn Ala Met Asp Val
Val Leu Gln Pro Ser Pro Ala Leu Ser Trp Arg Ser Thr Gly Gly Ile
Leu Asp Val Tyr Ile Phe Leu Gly Pro Glu Pro Lys Ser Val Val Gln
Gln Tyr Leu Asp Val Val Gly Tyr Pro Phe Met Pro Pro Tyr Trp Gly
Leu Gly Phe His Leu Cys Arg Trp Gly Tyr Ser Ser Thr Ala Ile Thr
Arg Gln Val Val Glu Asn Met Thr Arg Ala His Phe Pro Leu Asp Val
Gln Trp Asn Asp Leu Asp Tyr Met Asp Ser Arg Arg Asp Phe Thr
Phe Asn Lys Asp Gly Phe Arg Asp Phe Pro Ala Met Val Gln Glu
Leu His Gln Gly Gly Arg Arg Tyr Met Met Ile Val Asp Pro Ala Ile
Ser Ser Ser Gly Pro Ala Gly Ser Tyr Arg Pro Tyr Asp Glu Gly Leu
Arg Arg Gly Val Phe Ile Thr Asn Glu Thr Gly Gln Pro Leu Ile Gly
Lys Val Trp Pro Gly Ser Thr Ala Phe Pro Asp Phe Thr Asn Pro Thr
Ala Leu Ala Trp Trp Glu Asp Met Val Ala Glu Phe His Asp Gln Val
Pro Phe Asp Gly Met Trp Ile Asp Met Asn Glu Pro Ser Asn Phe Ile
Arg Gly Ser Glu Asp Gly Cys Pro Asn Asn Glu Leu Glu Asn Pro Pro
Tyr Val Pro Gly Val Val Gly Gly Thr Leu Gln Ala Ala Thr Ile Cys
Ala Ser Ser His Gln Phe Leu Ser Thr His Tyr Asn Leu His Asn Leu
Tyr Gly Leu Thr Glu Ala Ile Ala Ser His Arg Ala Leu Val Lys Ala
Arg Gly Thr Arg Pro Phe Val Ile Ser Arg Ser Thr Phe Ala Gly His
Gly Arg Tyr Ala Gly His Trp Thr Gly Asp Val Trp Ser Ser Trp Glu
Gln Leu Ala Ser Ser Val Pro Glu Ile Leu Gln Phe Asn Leu Leu Gly
Val Pro Leu Val Gly Ala Asp Val Cys Gly Phe Leu Gly Asn Thr Ser
Glu Glu Leu Cys Val Arg Trp Thr Gln Leu Gly Ala Phe Tyr Pro Phe
Met Arg Asn His Asn Ser Leu Leu Ser Leu Pro Gln Glu Pro Tyr Ser
Phe Ser Glu Pro Ala Gln Gln Ala Met Arg Lys Ala Leu Thr Leu Arg
Tyr Ala Leu Leu Pro His Leu Tyr Thr Leu Phe His Gln Ala His Val
Ala Gly Glu Thr Val Ala Arg Pro Leu Phe Leu Glu Phe Pro Lys Asp
Ser Ser Thr Trp Thr Val Asp His Gln Leu Leu Trp Gly Glu Ala Leu
Leu Ile Thr Pro Val Leu Gln Ala Gly Lys Ala Glu Val Thr Gly Tyr
Phe Pro Leu Gly Thr Trp Tyr Asp Leu Gln Thr Val Pro Ile Glu Ala
Leu Gly Ser Leu Pro Pro Pro Pro Ala Ala Pro Arg Glu Pro Ala Ile
His Ser Glu Gly Gln Trp Val Thr Leu Pro Ala Pro Leu Asp Thr Ile
Asn Val His Leu Arg Ala Gly Tyr Ile Ile Pro Leu Gln Gly Pro Gly
Leu Thr Thr Thr Glu Ser Arg Gln Gln Pro Met Ala Leu Ala Val Ala
Leu Thr Lys Gly Gly Glu Ala Arg Gly Glu Leu Phe Trp Asp Asp Gly
Glu Ser Leu Glu Val Leu Glu Arg Gly Ala Tyr Thr Gln Val Ile Phe
Leu Ala Arg Asn Asn Thr Ile Val Asn Glu Leu Val Arg Val Thr Ser
Glu Gly Ala Gly Leu Gln Leu Gln Lys Val Thr Val Leu Gly Val Ala
Thr Ala Pro Gln Gln Val Leu Ser Asn Gly Val Pro Val Ser Asn Phe
Thr Tyr Ser Pro Asp Thr Lys Val Leu Asp Ile Cys Val Ser Leu Leu
Met Gly Glu Gln Phe Leu Val Ser Trp Cys
SEQ ID NO: 2
Gln Gln Gly Ala Ser Arg Pro Gly Pro Arg Asp Ala Gln Ala His Pro
Gly Arg Pro Arg Ala Val Pro Thr Gln Cys Asp Val Pro Pro Asn Ser
Arg Phe Asp Cys Ala Pro Asp Lys Ala Ile Thr Gln Glu Gln Cys Glu
Ala Arg Gly Cys Cys Tyr Ile Pro Ala Lys Gln Gly Leu Gln Gly Ala
Gln Met Gly Gln Pro Trp Cys Phe Phe Pro Pro Ser Tyr Pro Ser Tyr
Lys Leu Glu Asn Leu Ser Ser Ser Glu Met Gly Tyr Thr Ala Thr Leu
Thr Arg Thr Thr Pro Thr Phe Phe Pro Lys Asp Ile Leu Thr Leu Arg
Leu Asp Val Met Met Glu Thr Glu Asn Arg Leu His Phe Thr Ile Lys
Asp Pro Ala Asn Arg Arg Tyr Glu Val Pro Leu Glu Thr Pro Arg Val
His Ser Arg Ala Pro Ser Pro Leu Tyr Ser Val Glu Phe Ser Glu Glu
Pro Phe Gly Val Ile Val His Arg Gln Leu Asp Gly Arg Val Leu Leu
Asn Thr Thr Val Ala Pro Leu Phe Phe Ala Asp Gln Phe Leu Gln Leu
Ser Thr Ser Leu Pro Ser Gln Tyr Ile Thr Gly Leu Ala Glu His Leu
Ser Pro Leu Met Leu Ser Thr Ser Trp Thr Arg Ile Thr Leu Trp Asn
Arg Asp Leu Ala Pro Thr Pro Gly Ala Asn Leu Tyr Gly Ser His Pro
Phe Tyr Leu Ala Leu Glu Asp Gly Gly Ser Ala His Gly Val Phe Leu
Leu Asn Ser Asn Ala Met Asp Val Val Leu Gln Pro Ser Pro Ala Leu
Ser Trp Arg Ser Thr Gly Gly Ile Leu Asp Val Tyr Ile Phe Leu Gly
Pro Glu Pro Lys Ser Val Val Gln Gln Tyr Leu Asp Val Val Gly Tyr
Pro Phe Met Pro Pro Tyr Trp Gly Leu Gly Phe His Leu Cys Arg Trp
Gly Tyr Ser Ser Thr Ala Ile Thr Arg Gln Val Val Glu Asn Met Thr
Arg Ala His Phe Pro Leu Asp Val Gln Trp Asn Asp Leu Asp Tyr
Met Asp Ser Arg Arg Asp Phe Thr Phe Asn Lys Asp Gly Phe Arg
Asp Phe Pro Ala Met Val Gln Glu Leu His Gln Gly Gly Arg Arg Tyr
Met Met Ile Val Asp Pro Ala Ile Ser Ser Ser Gly Pro Ala Gly Ser Tyr
Arg Pro Tyr Asp Glu Gly Leu Arg Arg Gly Val Phe Ile Thr Asn Glu
Thr Gly Gln Pro Leu Ile Gly Lys Val Trp Pro Gly Ser Thr Ala Phe
Pro Asp Phe Thr Asn Pro Thr Ala Leu Ala Trp Trp Glu Asp Met Val
Ala Glu Phe His Asp Gln Val Pro Phe Asp Gly Met Trp Ile Asp Met
Asn Glu Pro Ser Asn Phe Ile Arg Gly Ser Glu Asp Gly Cys Pro Asn
Asn Glu Leu Glu Asn Pro Pro Tyr Val Pro Gly Val Val Gly Gly Thr
Leu Gln Ala Ala Thr Ile Cys Ala Ser Ser His Gln Phe Leu Ser Thr
His Tyr Asn Leu His Asn Leu Tyr Gly Leu Thr Glu Ala Ile Ala Ser
His Arg Ala Leu Val Lys Ala Arg Gly Thr Arg Pro Phe Val Ile Ser
Arg Ser Thr Phe Ala Gly His Gly Arg Tyr Ala Gly His Trp Thr Gly
Asp Val Trp Ser Ser Trp Glu Gln Leu Ala Ser Ser Val Pro Glu Ile
Leu Gln Phe Asn Leu Leu Gly Val Pro Leu Val Gly Ala Asp Val Cys
Gly Phe Leu Gly Asn Thr Ser Glu Glu Leu Cys Val Arg Trp Thr Gln
Leu Gly Ala Phe Tyr Pro Phe Met Arg Asn His Asn Ser Leu Leu Ser
Leu Pro Gln Glu Pro Tyr Ser Phe Ser Glu Pro Ala Gln Gln Ala Met
Arg Lys Ala Leu Thr Leu Arg Tyr Ala Leu Leu Pro His Leu Tyr Thr
Leu Phe His Gln Ala His Val Ala Gly Glu Thr Val Ala Arg Pro Leu
Phe Leu Glu Phe Pro Lys Asp Ser Ser Thr Trp Thr Val Asp His Gln
Leu Leu Trp Gly Glu Ala Leu Leu Ile Thr Pro Val Leu Gln Ala Gly
Lys Ala Glu Val Thr Gly Tyr Phe Pro Leu Gly Thr Trp Tyr Asp Leu
Gln Thr Val Pro Ile Glu Ala Leu Gly Ser Leu Pro Pro Pro Pro Ala
Ala Pro Arg Glu Pro Ala Ile His Ser Glu Gly Gln Trp Val Thr Leu
Pro Ala Pro Leu Asp Thr Ile Asn Val His Leu Arg Ala Gly Tyr Ile Ile
Pro Leu Gln Gly Pro Gly Leu Thr Thr Thr Glu Ser Arg Gln Gln Pro
Met Ala Leu Ala Val Ala Leu Thr Lys Gly Gly Glu Ala Arg Gly Glu
Leu Phe Trp Asp Asp Gly Glu Ser Leu Glu Val Leu Glu Arg Gly Ala
Tyr Thr Gln Val Ile Phe Leu Ala Arg Asn Asn Thr Ile Val Asn Glu
Leu Val Arg Val Thr Ser Glu Gly Ala Gly Leu Gln Leu Gln Lys Val
Thr Val Leu Gly Val Ala Thr Ala Pro Gln Gln Val Leu Ser Asn Gly
Val Pro Val Ser Asn Phe Thr Tyr Ser Pro Asp Thr Lys Val Leu Asp
Ile Cys Val Ser Leu Leu Met Gly Glu Gln Phe Leu Val Ser Trp Cys

In at least one embodiment, the recombinant human acid α-glucosidase has a wild-type GAA amino acid sequence as set forth in SEQ ID NO: 1, as described in U.S. Pat. No. 8,592,362 and has GenBank accession number AHE24104.1 (GI:568760974). In at least one embodiment, the recombinant human acid α-glucosidase is glucosidase alfa, the human acid α-glucosidase enzyme encoded by the most predominant of nine observed haplotypes of the GAA gene.

In at least one embodiment, the recombinant human acid α-glucosidase is initially expressed as having the full-length 952 amino acid sequence of wild-type GAA as set forth in SEQ ID NO: 1, and the recombinant human acid α-glucosidase undergoes intracellular processing that removes a portion of the amino acids, e.g. the first 56 amino acids. Accordingly, the recombinant human acid α-glucosidase that is secreted by the host cell can have a shorter amino acid sequence than the recombinant human acid α-glucosidase that is initially expressed within the cell. In at least one embodiment, the shorter protein can have the amino acid sequence set forth in SEQ ID NO: 2, which only differs from SEQ ID NO: 1 in that the first 56 amino acids comprising the signal peptide and precursor peptide have been removed, thus resulting in a protein having 896 amino acids. Other variations in the number of amino acids is also possible, such as having 1, 2, 3, 4, 5, 6, 7, 8, 9, 10, 11, 12, 13, 14, 15 or more deletions, substitutions and/or insertions relative to the amino acid sequence described by SEQ ID NO: 1 or SEQ ID NO: 2. In some embodiments, the rhGAA product includes a mixture of recombinant human acid α-glucosidase molecules having different amino acid lengths.

In at least one embodiment, the recombinant human acid α-glucosidase undergoes post-translational and/or chemical modifications at one or more amino acid residues in the protein. For example, methionine and tryptophan residues can undergo oxidation. As another example, the N-terminal glutamine can form pyro-glutamate. As another example, asparagine residues can undergo deamidation to aspartic acid. As yet another example, aspartic acid residues can undergo isomerization to iso-aspartic acid. As yet another example, unpaired cysteine residues in the protein can form disulfide bonds with free glutathione and/or cysteine. Accordingly, in some embodiments the enzyme is initially expressed as having an amino acid sequence as set forth in SEQ ID NO: 1 or SEQ ID NO: 2, and the enzyme undergoes one or more of these post-translational and/or chemical modifications. Such modifications are also within the scope of the present disclosure.

Polynucleotide sequences encoding GAA and such variant human GAAs are also contemplated and may be used to recombinantly express rhGAAs according to the invention.

Preferably, no more than 70, 65, 60, 55, 45, 40, 35, 30, 25, 20, 15, 10, or 5% of the total recombinant protein (e.g. rhGAA) molecules lack an N-glycan unit bearing one or more mannose-6-phosphate residues or lacks a capacity to bind to the cation independent mannose-6-phosphate receptor (CIMPR). Alternatively, 30, 35, 40, 45, 50, 55, 60, 65, 70, 75, 80, 85, 90, 95, 99%, <100% or more of the recombinant protein (e.g. rhGAA) molecules comprise at least one N-glycan unit bearing one or more mannose-6-phosphate residues or has the capacity to bind to CIMPR.

The recombinant protein (e.g. rhGAA) molecules may have 1, 2, 3 or 4 mannose-6-phosphate (M6P) groups on their glycans. For example, only one N-glycan on a recombinant protein molecule may bear M6P (mono-phosphorylated), a single N-glycan may bear two M6P groups (bis-phosphorylated), or two different N-glycans on the same recombinant protein molecule may each bear single M6P groups. Recombinant protein molecules may also have N-glycans bearing no M6P groups. In another embodiment, on average the N-glycans contain greater than 3 mol/mol of M6P and greater than 4 mol/mol sialic acid, such that the recombinant protein comprises on average at least 3 moles of mannose-6-phosphate residues per mole of recombinant protein and at least 4 moles of sialic acid per mole of recombinant protein. On average at least about 3, 4, 5, 6, 7, 8, 9, or 10% of the total glycans on the recombinant protein may be in the form of a mono-M6P glycan, for example, about 6.25% of the total glycans may carry a single M6P group and on average, at least about 0.5, 1, 1.5, 2.0, 2.5, 3.0% of the total glycans on recombinant protein are in the form of a bis-M6P glycan and on average less than 25% of total recombinant protein contains no phosphorylated glycan binding to CIMPR.

The recombinant protein (e.g. rhGAA) may have an average content of N-glycans carrying M6P ranging from 0.5 to 7.0 mol/mol lysosomal protein or any intermediate value of subrange including 0.5, 1.0, 1.5, 2.0, 2.5, 3.0, 3.5, 4.0, 4.5, 5.0, 5.5, 6.0, 6.5, or 7.0 mol/mol lysosomal protein. The lysosomal protein can be fractionated to provide lysosomal protein preparations with different average numbers of M6P-bearing or bis-M6P-bearing glycans thus permitting further customization of lysosomal protein targeting to the lysosomes in target tissues by selecting a particular fraction or by selectively combining different fractions.

In some embodiments, the recombinant protein (e.g. rhGAA) will bear, on average, 2.0 to 8.0 moles of M6P per mole of recombinant protein (e.g. rhGAA). This range includes all intermediate values and subranges including 2.0, 2.5, 3.0, 3.5, 4.0, 4.5, 5.0, 5.5, 6.0, 6.5, 7.0, 7.5 and 8.0 mol M6P/mol recombinant protein (e.g. rhGAA).

Up to 60% of the N-glycans on the recombinant protein (e.g. rhGAA) may be fully sialylated, for example, up to 10%, 20%, 30%, 40%, 50% or 60% of the N-glycans may be fully sialylated. In some embodiments from 4 to 20% of the total N-glycans are fully sialylated. In other embodiments no more than 5%, 10%, 20% or 30% of N-glycans on the recombinant protein (e.g. rhGAA) carry sialic acid and a terminal galactose residue (Gal). This range includes all intermediate values and subranges, for example, 7 to 30% of the total N-glycans on the recombinant protein can carry sialic acid and terminal galactose. In yet other embodiments, no more than 5, 10, 15, 16, 17, 18, 19 or 20% of the N-glycans on recombinant protein have a terminal galactose only and do not contain sialic acid. This range includes all intermediate values and subranges, for example, from 8 to 19% of the total N-glycans on the recombinant protein in the composition may have terminal galactose only and do not contain sialic acid.

In other embodiments of the invention, 40, 45, 50, 55 to 60% of the total

N-glycans on the recombinant protein (e.g. rhGAA) are complex type N-glycans; or no more than 1, 2, 3, 4, 5, 6, 7% of total N-glycans on the recombinant protein (e.g. rhGAA) are hybrid-type N-glycans; no more than 5, 10, or 15% of the high mannose-type N-glycans on the recombinant protein (e.g. rhGAA) are non-phosphorylated; at least 5% or 10% of the high mannose-type N-glycans on the recombinant protein (e.g. rhGAA) are mono-M6P phosphorylated; and/or at least 1 or 2% of the high mannose-type N-glycans on the recombinant protein (e.g. rhGAA) are bis-M6P phosphorylated. These values include all intermediate values and subranges. A recombinant protein (e.g. rhGAA) may meet one or more of the content ranges described above.

In some embodiments, the recombinant protein (e.g. rhGAA) will bear, on average, 2.0 to 8.0 moles of sialic acid residues per mole of recombinant protein (e.g. rhGAA). This range includes all intermediate values and subranges including 2.0, 2.5, 3.0, 3.5, 4.0, 4.5, 5.0, 5.5, 6.0, 6.5, 7.0, 7.5 and 8.0 mol residues/mol recombinant protein (e.g. rhGAA). Without being bound by theory, it is believed that the presence of N-glycan units bearing sialic acid residues may prevent non-productive clearance of the recombinant protein (e.g. rhGAA) by asialoglycoprotein receptors.

In one or more embodiments, the recombinant protein (e.g. rhGAA) has M6P and/or sialic acid units at certain N-glycosylation sites of the recombinant protein. For example, as stated above, there are seven potential N-linked glycosylation sites on rhGAA. These potential glycosylation sites are at the following positions of SEQ ID NO: 2: N84, N177, N334, N414, N596, N826 and N869. Similarly, for the full-length amino acid sequence of SEQ ID NO: 1, these potential glycosylation sites are at the following positions: N140, N233, N390, N470, N652, N882 and N925. Other variants of rhGAA can have similar glycosylation sites, depending on the location of asparagine residues. Generally, sequences of ASN-X-SER or ASN-X-THR in the protein amino acid sequence indicate potential glycosylation sites, with the exception that X cannot be HIS or PRO.

In various embodiments, the rhGAA has a certain N-glycosylation profile. In one or more embodiments, at least 20% of the rhGAA is phosphorylated at the first N-glycosylation site (e.g. N84 for SEQ ID NO: 2 and N140 for SEQ ID NO: 1). For example, at least 20%, 25%, 30%, 35%, 40%, 45%, 50%, 55%, 60%, 65%, 70%, 75%, 80%, 85%, 90% or 95% of the rhGAA can be phosphorylated at the first N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a mono-M6P unit at the first N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a bis-M6P unit at the first N-glycosylation site.

In one or more embodiments, at least 20% of the rhGAA is phosphorylated at the second N-glycosylation site (e.g. N177 for SEQ ID NO: 2 and N223 for SEQ ID NO: 1). For example, at least 20%, 25%, 30%, 35%, 40%, 45%, 50%, 55%, 60%, 65%, 70%, 75%, 80%, 85%, 90% or 95% of the rhGAA can be phosphorylated at the second N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a mono-M6P unit at the second N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a bis-M6P unit at the second N-glycosylation site. In one or more embodiments, at least 5% of the rhGAA is phosphorylated at the third N-glycosylation site (e.g. N334 for SEQ ID NO: 2 and N390 for SEQ ID NO: 1). In other embodiments, less than 5%, 10%, 15%, 20% or 25% of the rhGAA is phosphorylated at the third N-glycosylation site. For example, the third N-glycosylation site can have a mixture of non-phosphorylated high mannose glycans, di-, tri-, and tetra-antennary complex glycans, and hybrid glycans as the major species. In some embodiments, at least 3%, 5%, 8%, 10%, 15%, 20%, 25%, 30%, 35%, 40%, 45% or 50% of the rhGAA is sialylated at the third N-glycosylation site.

In one or more embodiments, at least 20% of the rhGAA is phosphorylated at the fourth N-glycosylation site (e.g. N414 for SEQ ID NO: 2 and N470 for SEQ ID NO: 1). For example, at least 20%, 25%, 30%, 35%, 40%, 45%, 50%, 55%, 60%, 65%, 70%, 75%, 80%, 85%, 90% or 95% of the rhGAA can be phosphorylated at the fourth N-glycosylation site. This phosphorylation can be the result of mono-M6P and/or bis-M6P units. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a mono-M6P unit at the fourth N-glycosylation site. In some embodiments, at least 10%, 15%, 20%, 25%, 30%, 35%, 40%, 45%, 50%, 55%, 60%, 65%, 70%, 75%, 80%, 85%, 90% or 95% of the rhGAA bears a bis-M6P unit at the fourth N-glycosylation site. In some embodiments, at least 3%, 5%, 8%, 10%, 15%, 20% or 25% of the rhGAA is sialylated at the fourth N-glycosylation site.

In one or more embodiments, at least 5% of the rhGAA is phosphorylated at the fifth N-glycosylation site (e.g. N596 for SEQ ID NO: 2 and N692 for SEQ ID NO: 1). In other embodiments, less than 5%, 10%, 15%, 20% or 25% of the rhGAA is phosphorylated at the fifth N-glycosylation site. For example, the fifth N-glycosylation site can have fucosylated di-antennary complex glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90% or 95% of the rhGAA is sialylated at the fifth N-glycosylation site.

In one or more embodiments, at least 5% of the rhGAA is phosphorylated at the sixth N-glycosylation site (e.g. N826 for SEQ ID NO: 2 and N882 for SEQ ID NO: 1). In other embodiments, less than 5%, 10%, 15%, 20% or 25% of the rhGAA is phosphorylated at the sixth N-glycosylation site. For example, the sixth N-glycosylation site can have a mixture of di-, tri-, and tetra-antennary complex glycans as the major species. In some embodiments, at least 3%, 5%, 8%, 10%, 15%, 20%, 25%, 30%, 35%, 40%, 45%, 50%, 55%, 60%, 65%, 70%, 75%, 80%, 85%, 90% or 95% of the rhGAA is sialylated at the sixth N-glycosylation site.

In one or more embodiments, at least 5% of the rhGAA is phosphorylated at the seventh N-glycosylation site (e.g. N869 for SEQ ID NO: 2 and N925 for SEQ ID NO: 1). In other embodiments, less than 5%, 10%, 15%, 20% or 25% of the rhGAA is phosphorylated at the seventh N-glycosylation site. In some embodiments, less than 40%, 45%, 50%, 55%, 60% or 65% % of the rhGAA has any glycan at the seventh N-glycosylation site. In some embodiments, at least 30%, 35% or 40% of the rhGAA has a glycan at the seventh N-glycosylation site.

In one or more embodiments, 40%-60% of the N-glycans on the rhGAA are complex type N-glycans; and the rhGAA comprises 3.0-5.0 mol M6P residues per mol rhGAA.

In various embodiments, the rhGAA has an average fucose content of 0-5 mol per mol of rhGAA, GlcNAc content of 10-30 mol per mol of rhGAA, galactose content of 5-20 mol per mol of rhGAA, mannose content of 10-40 mol per mol of rhGAA, M6P content of 2-8 mol per mol of rhGAA and sialic acid content of 2-8 mol per mol of rhGAA. In various embodiments, the rhGAA has an average fucose content of 2-3 mol per mol of rhGAA, GlcNAc content of 20-25 mol per mol of rhGAA, galactose content of 8-12 mol per mol of rhGAA, mannose content of 22-27 mol per mol of rhGAA, M6P content of 3-5 mol per mol of rhGAA and sialic acid content of 4-7 mol of rhGAA.

The recombinant protein (e.g. rhGAA) is preferably produced by Chinese hamster ovary (CHO) cells, such as CHO cell line GA-ATB-200 or ATB-200-001-X5-14, or by a subculture or derivative of such a CHO cell culture. DNA constructs, which express allelic variants of acid α-glucosidase or other variant acid α-glucosidase amino acid sequences such as those that are at least 90%, 95%, 98% or 99% identical to SEQ ID NO: 1 or SEQ ID NO: 2, may be constructed and expressed in CHO cells. These variant acid α-glucosidase amino acid sequences may contain deletions, substitutions and/or insertions relative to SEQ ID NO: 1 or SEQ ID NO: 2, such as having 1, 2, 3, 4, 5, 6, 7, 8, 9, 10, 11, 12, 13, 14, 15 or more deletions, substitutions and/or insertions relative to the amino acid sequence described by SEQ ID NO: 1 or SEQ ID NO: 2. Those of skill in the art can select alternative vectors suitable for transforming CHO cells for production of such DNA constructs.

Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

As described in U.S. Pat. No. 10,208,299, recombinant human acid α-glucosidase having superior ability to target cation-independent mannose-6-phosphate receptors (CIMPR) and cellular lysosomes as well as glycosylation patterns that reduce its non-productive clearance in vivo can be produced using Chinese hamster ovary (CHO) cells. These cells can be induced to express recombinant human acid α-glucosidase with significantly higher levels of N-glycan units bearing one or more mannose-6-phosphate residues than conventional recombinant human acid α-glucosidase products such as alglucosidase alfa. The recombinant human acid α-glucosidase produced by these cells, for example, as exemplified by ATB200, has significantly more muscle cell-targeting mannose-6-phosphate (M6P) and bis-mannose-6-phosphate N-glycan residues than conventional acid α-glucosidase, such as Lumizyme®. Without being bound by theory, it is believed that this extensive glycosylation allows the ATB200 enzyme to be taken up more effectively into target cells, and therefore to be cleared from the circulation more efficiently than other recombinant human acid α-glucosidases, such as for example, alglucosidase alfa, which has a much lower M6P and bis-M6P content. ATB200 has been shown to efficiently bind to CIMPR and be efficiently taken up by skeletal muscle and cardiac muscle and to have a glycosylation pattern that provides a favorable pharmacokinetic profile and reduces non-productive clearance in vivo.

It is also contemplated that the extensive glycosylation of ATB200 can contribute to a reduction of the immunogenicity of ATB200 compared to, for example, alglucosidase alfa. As will be appreciated by those skilled in the art, glycosylation of proteins with conserved mammalian sugars generally enhances product solubility and diminishes product aggregation and immunogenicity. Glycosylation indirectly alters protein immunogenicity by minimizing protein aggregation as well as by shielding immunogenic protein epitopes from the immune system (Guidance for Industry-Immunogenicity

Assessment for Therapeutic Protein Products, US Department of Health and Human Services, Food and Drug Administration, Center for Drug Evaluation and Research, Center for Biologics Evaluation and Research, August 2014). Therefore, in at least one embodiment, administration of the recombinant human acid α-glucosidase does not induce anti-drug antibodies. In at least one embodiment, administration of the recombinant human acid α-glucosidase induces a lower incidence of anti-drug antibodies in a subject than the level of anti-drug antibodies induced by administration of alglucosidase alfa.

As described in U.S. Pat. No. 10,208,299, cells such as CHO cells can be used to produce the rhGAA described therein, and this rhGAA can be used in the present invention. Examples of such a CHO cell line are GA-ATB-200 or ATB-200-001-X5-14, or a subculture thereof that produces a rhGAA composition as described therein. Such CHO cell lines may contain multiple copies of a gene, such as 5, 10, 15, or 20 or more copies, of a polynucleotide encoding GAA.

The high M6P and bis-M6P rhGAA, such as ATB200 rhGAA, can be produced by transforming CHO cells with a DNA construct that encodes GAA. While CHO cells have been previously used to make rhGAA, it was not appreciated that transformed CHO cells could be cultured and selected in a way that would produce rhGAA having a high content of M6P and bis-M6P glycans which target the CIMPR.

Surprisingly, it was found that it was possible to transform CHO cell lines, select transformants that produce rhGAA containing a high content of glycans bearing M6P or bis-M6P that target the CIMPR, and to stably express this high-M6P rhGAA. Thus, methods for making these CHO cell lines are also described in U.S. Pat. No. 10,208,299. This method involves transforming a CHO cell with DNA encoding GAA or a GAA variant, selecting a CHO cell that stably integrates the DNA encoding GAA into its chromosome(s) and that stably expresses GAA, and selecting a CHO cell that expresses GAA having a high content of glycans bearing M6P or bis-M6P, and, optionally, selecting a CHO cell having N-glycans with high sialic acid content and/or having N-glycans with a low non-phosphorylated high-mannose content.

These CHO cell lines may be used to produce rhGAA and rhGAA compositions by culturing the CHO cell line and recovering said composition from the culture of CHO cells.

### Production, Capturing and Purification of Biologics

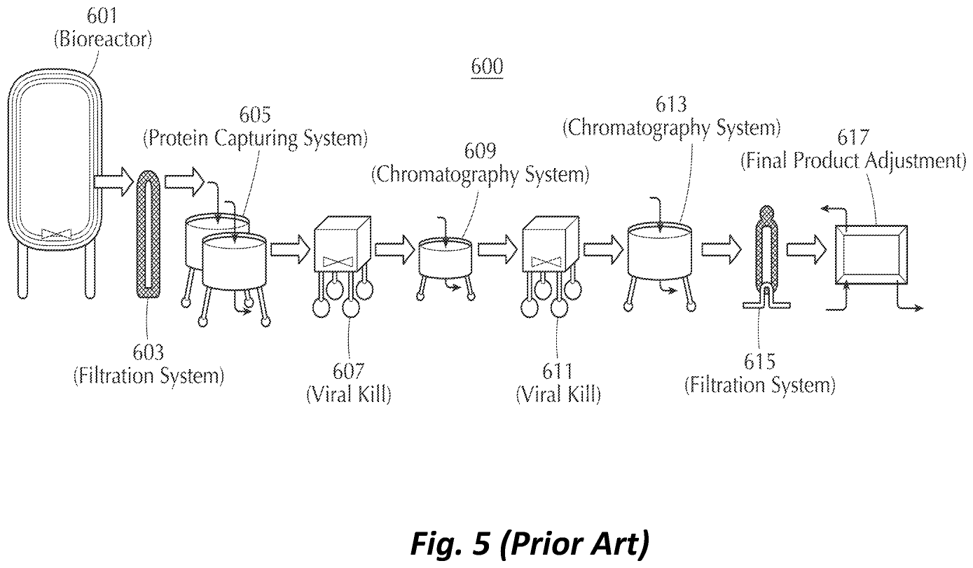

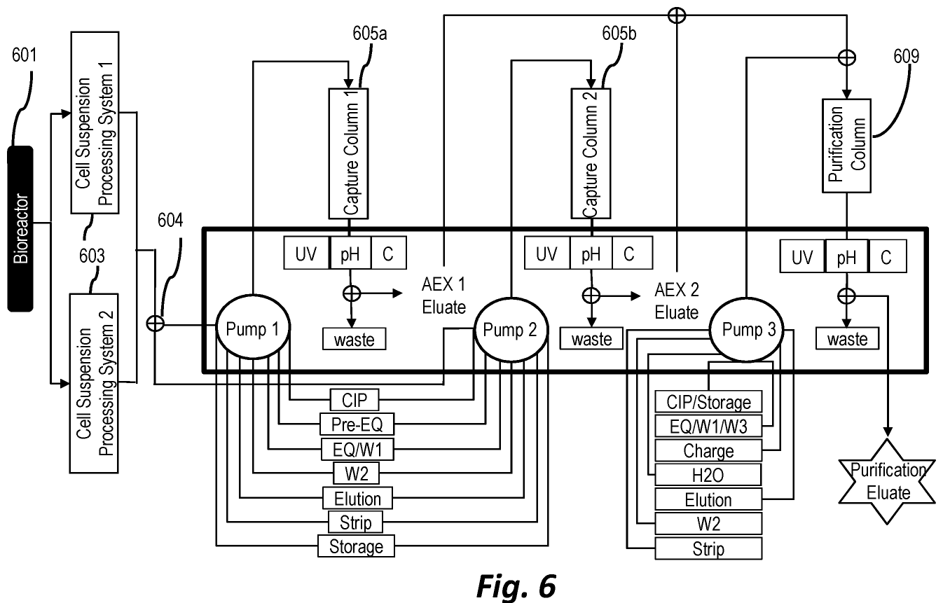

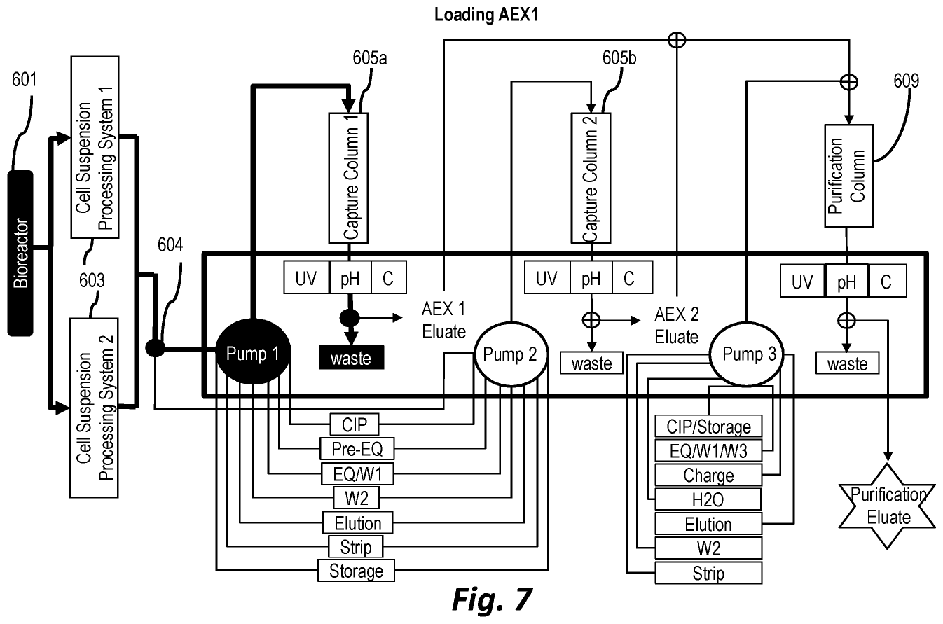

Various embodiments of the present invention pertain to methods for the production and/or capturing and/or purification of biologics (e.g. recombinant proteins including recombinant human lysosomal protein such as rhGAA, antibodies and virus particles). An exemplary prior art process 600 for producing, capturing and purifying biologics is shown in FIG. 5. Exemplary processes for producing, capturing and purifying biologics according to one or more embodiments of the invention are shown in FIGS. 6 and 7. FIG. 6 shows a configuration with two capture columns (e.g. AEX columns) and one purification column (e.g. IMAC column), whereas FIG. 7 shows a configuration with two capture columns (e.g. AEX columns) and two purification columns (e.g. IMAC columns).

In FIGS. 5-13, the arrows indicate the direction of movement for the various liquid phases containing the biologics (e.g. recombinant human lysosomal protein such as rhGAA). Bioreactor 601 contains a culture of cells, such as CHO cells, that produce biologics (e.g. rhGAA). The biologics include recombinant proteins, antibodies and virus particles. The recombinant proteins may be a secreted protein, membrane protein or intracellular protein. The bioreactor 601 can be any appropriate bioreactor for culturing the cells, such as a perfusion, batch or fed-batch bioreactor. In various embodiments, the bioreactor has a volume between about 1 L and about 20,000 L. Exemplary bioreactor volumes include about 1 L, about 10 L, about 20 L, about 30 L, about 40 L, about 50 L, about 60 L, about 70 L, about 80 L, about 90 L, about 100 L, about 150 L, about 200 L, about 250 L, about 300 L, about 350 L, about 400 L, about 500 L, about 600 L, about 700 L, about 800 L, about 900 L, about 1,000 L, about 1,500 L, about 2,000 L, about 2,500 L, about 3,000 L, about 3,500 L, about 4,000 L, about 5,000 L, about 6,000 L, about 7,000 L, about 8,000 L, about 9,000 L, about 10,000 L, about 15,000 L and about 20,000 L.

As shown in FIGS. 5-13, the media and/or cell suspension can be removed from the bioreactor. Such removal can be continuous for a perfusion bioreactor or can be batchwise for a batch or fed-batch reactor. The media and/or cell suspension is processed to separate a filtrate containing the biologics via cell suspension processing system 603. In one or more embodiments, the cell suspension processing system includes one or more steps of cell lysis, filtration, centrifugation and membrane solublization. In some embodiments, the biologic is a secreted recombinant protein. In some embodiments, the cells removed from the media are re-introduced to the bioreactor and the media comprising the secreted recombinant protein can be further processed. In some embodiment, the cell suspension processing system 603 comprises a filtration system. The filtration system can be any suitable filtration system, including an alternating tangential flow filtration (ATF) system, a tangential flow filtration (TFF) system, centrifugal filtration system, etc. In various embodiments, the filtration system utilizes a filter having a pore size between about 10 nanometers and about 2 micrometers. Exemplary filter pore sizes include about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 350 nm, about 400 nm, about 500 nm, about 600 nm, about 700 nm, about 800 nm, about 900 nm, about 1 μm, about 1.5 μm and about 2 μm.

In various embodiments, the media and/or cell suspension removal rate is between about 1 L/day and about 20,000 L/day. Exemplary media and/or cell suspension removal rates include about 1 L/day, about 10 L/day, about 20 L/day, about 30 L/day, about 40 L/day, about 50 L/day, about 60 L/day, about 70 L/day, about 80 L/day, about 90 L/day, about 100 L/day, about 150 L/day, about 200 L/day, about 250 L/day, about 300 L/day, about 350

L/day, about 400 L/day, about 500 L/day, about 600 L/day, about 700 L/day, about 800 L/day, about 900 L/day, about 1,000 L/day, about 1,500 L/day, about 2,000 L/day, about 2,500 L/day, about 3,000 L/day, about 3,500 L/day, about 4,000 L/day, about 5,000 L/day, about 6,000 L/day, about 7,000 L/day, about 8,000 L/day, about 9,000 L/day, about 10,000 L/day, about 15,000 L/day and about 20,000 L/day. Alternatively, the media and/or cell suspension removal rate can be expressed as a function of the bioreactor volume, such as about 0.1 to about 3 reactor volumes per day. Exemplary media and/or cell suspension removal rates include about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 2, about 2.5 and about 3 reactor volumes per day.

For a continuous or fed-batch process, the rate at which fresh media is provided to the bioreactor can be between about 1 L/day and about 20,000 L/day. Exemplary media introduction rates include about 1 L/day, about 10 L/day, about 20 L/day, about 30 L/day, about 40 L/day, about 50 L/day, about 60 L/day, about 70 L/day, about 80 L/day, about 90 L/day, about 100 L/day, about 150 L/day, about 200 L/day, about 250 L/day, about 300 L/day, about 350 L/day, about 400 L/day, about 500 L/day, about 600 L/day, about 700 L/day, about 800 L/day, about 900 L/day, about 1,000 L/day, about 1,500 L/day, about 2,000 L/day, about 2,500 L/day, about 3,000 L/day, about 3,500 L/day, about 4,000 L/day, about 5,000 L/day, about 6,000 L/day, about 7,000 L/day, about 8,000 L/day, about 9,000 L/day, about 10,000 L/day, about 15,000 L/day and about 20,000 L/day. Alternatively, the media introduction rate can be expressed as a function of the bioreactor volume, such as about 0.1 to about 3 reactor volumes per day. Exemplary media introduction rates include about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 2, about 2.5 and about 3 reactor volumes per day.

After being processed through the cell suspension processing system, the collected filtrate is loaded onto a capturing system 605. The capturing system 605 can include one or more chromatography columns.

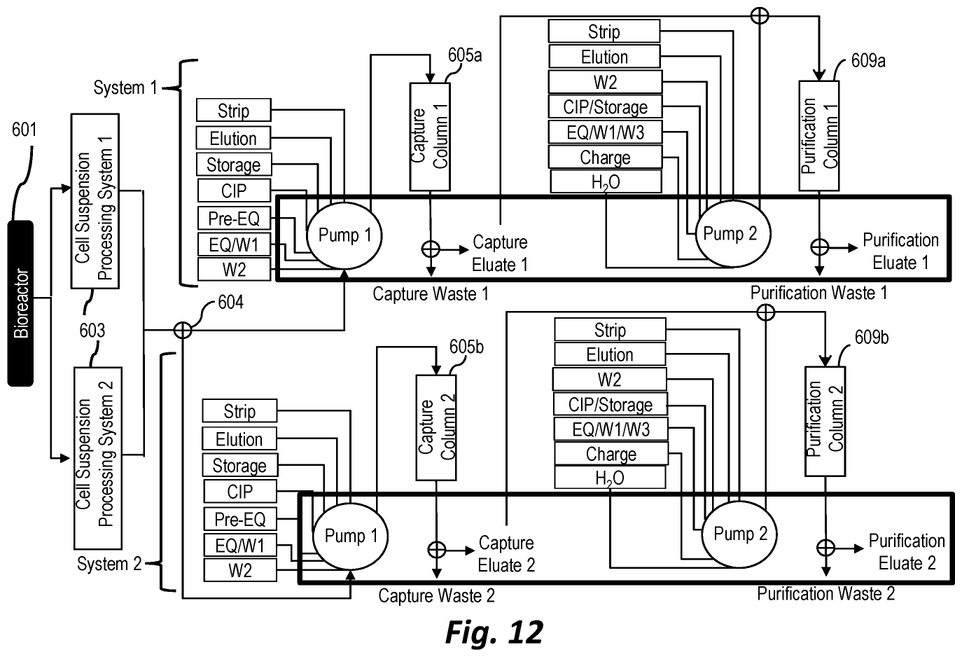

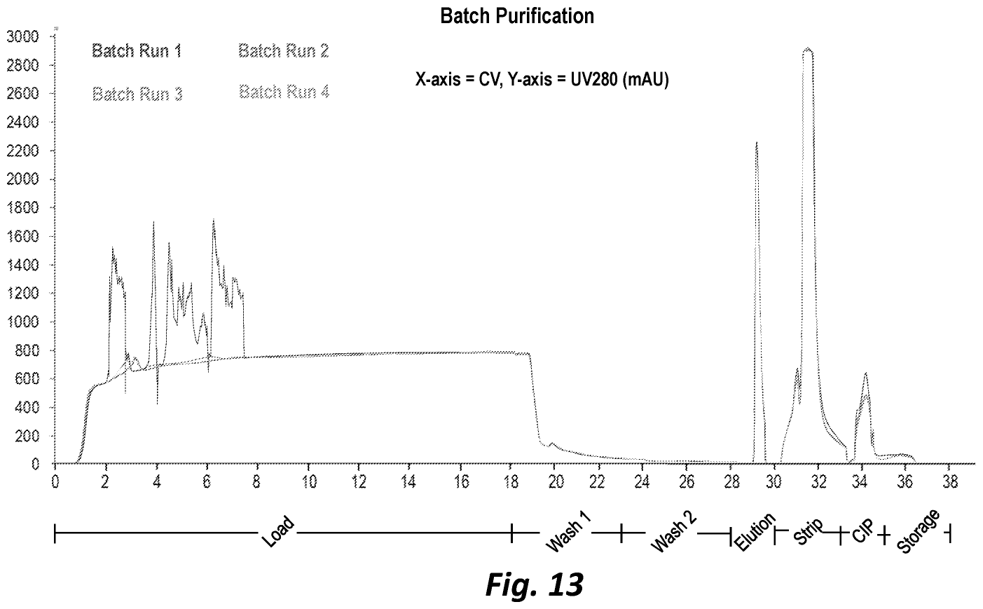

In FIG. 6, the capturing system 605 comprises two capture columns 605a and 605b. FIG. 13 shows two chromatography systems in which each system comprises a single capture column, i.e. capture column 605a is part of one chromatography system and capture column 605b is part of a separate, but identical, chromatography system. In both FIGS. 6 and 12, the capture columns are in parallel such that flowthrough of capture column 605a does not flow to capture 605b. Rather, once capture column 605a is loaded, valve 604 redirects the flow of filtrate to the second capture column 605b instead of capture column 605a. In one or more embodiments, the loading of capture columns 605a and 605b is cycled back and forth between the columns to provide for the continuous loading of media from the bioreactor onto the capture columns. If more than two capture columns are used, then the columns can be loaded sequentially or in different orders.

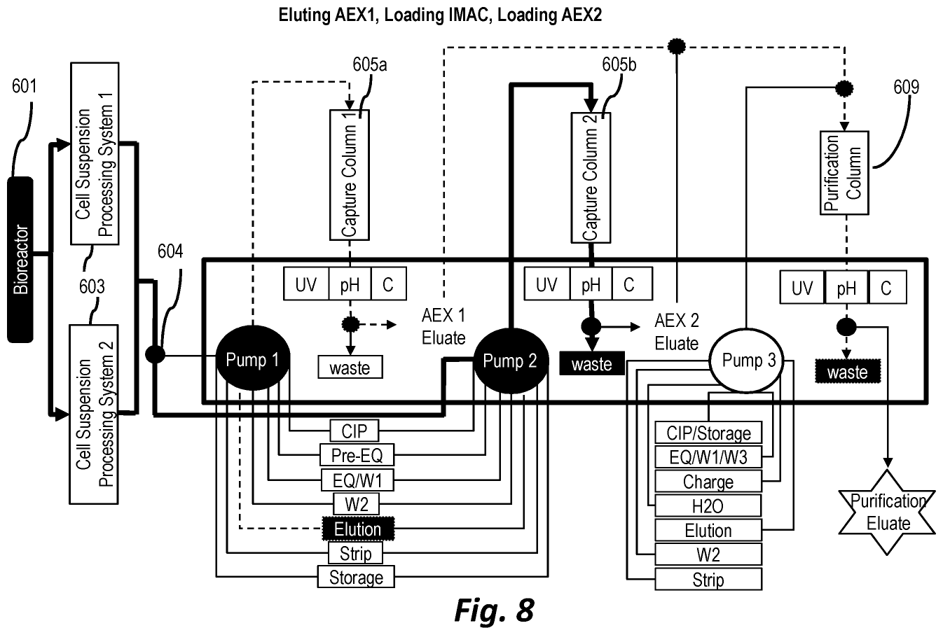

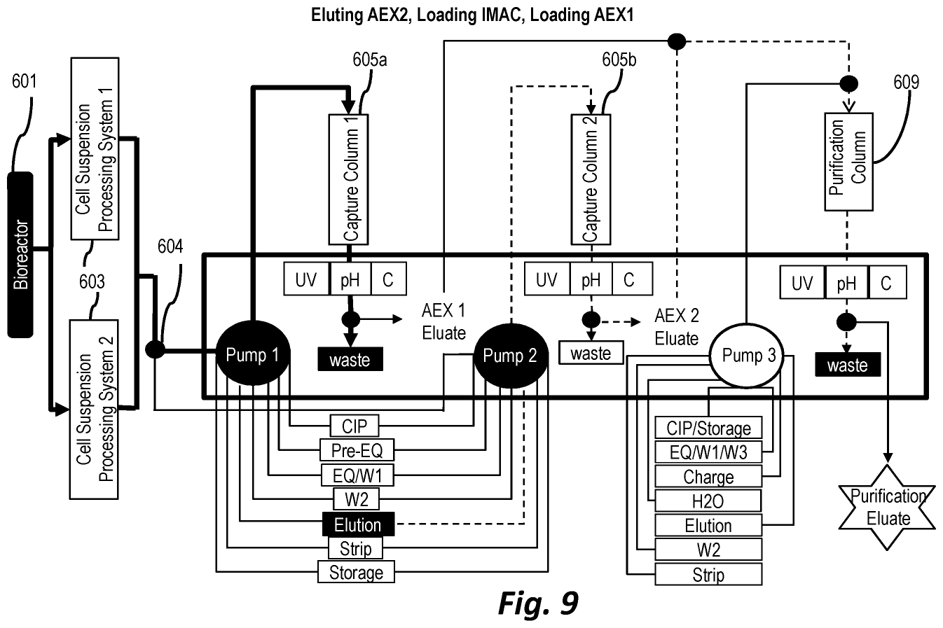

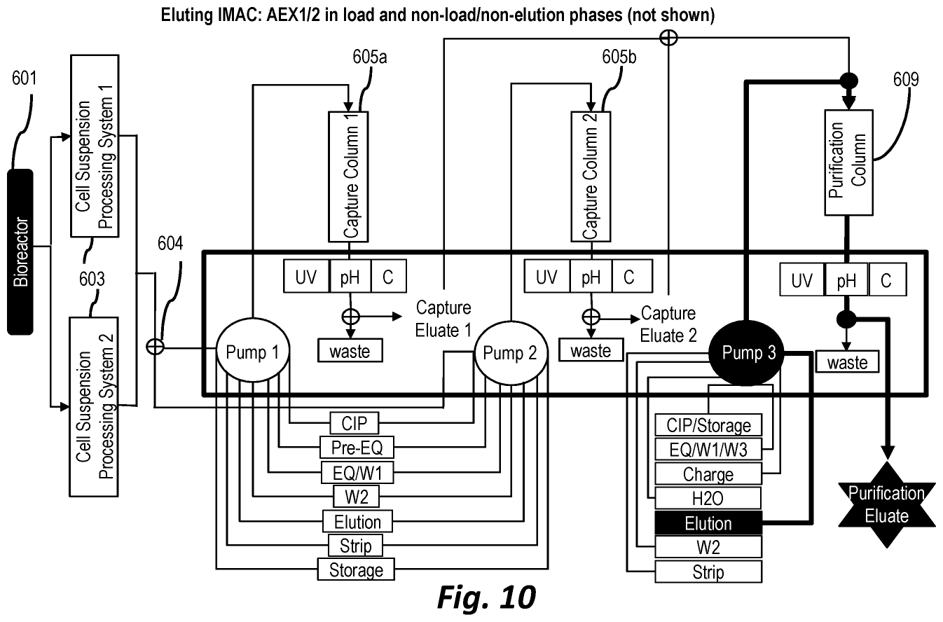

FIG. 7-10 describes two capture columns, capture column 1 and capture column 2, and a purification column working sequentially for continuous purification of the biologics. In FIG. 7, the capture column 1 is loaded with the filtrate containing the biologics. In FIG. 8, the captured biologic is eluted from the capture column 1 and loaded on the purification column while simultaneously the filtrate is loaded on the capture column 2. In FIG. 9, the captured biologic is eluted from the capture column 2 and loaded on the purification column while simultaneously the filtrate is loaded on the capture column 1. In FIG. 10, the purified biologic is eluted from the purification column.

In various embodiments, the capturing system 605 includes one or more capture columns (e.g. AEX) for the direct product capture of biologics. In some embodiments the capture column is an AEX column and the biologic is a secreted recombinant protein, particularly lysosomal protein having a high M6P content. While not wishing to be bound by any particular theory, it is believed that using AEX chromatography to capture the recombinant protein from the filtered media ensures that the captured recombinant protein product has a higher M6P content, due to the more negative charge of the recombinant protein having one or more M6P groups. As a result, non-phosphorylated recombinant protein and host cell impurities do not bind the column resin as well as the highly phosphorylated recombinant protein, and the non-phosphorylated recombinant protein and host cell impurities passes through the column. Accordingly, the AEX chromatography can be used to enrich the M6P content of the protein product (i.e. select for proteins having more M6P) due to the high affinity of the M6P-containing proteins for the AEX resin.

Furthermore, while not wishing to be bound by any particular theory, it is also believed that having a direct product capture of recombinant protein using AEX chromatography ensures that the recombinant proteins having high M6P content are removed from the media containing proteases and other enzymes that can degrade the protein and/or dephosphorylate the protein. As a result, the high quality product is preserved.

Suitable AEX chromatography columns have functional chemical groups that bind negatively charged molecules such as negatively charged proteins. Exemplary functional groups include, but are not limited to, primary, secondary, tertiary, and quaternary ammonium or amine groups. These functional groups may be bound to membranes (e.g. cellulose membranes) or conventional chromatography resins. Exemplary column media include SP, CM, Q and DEAE Sepharose® Fast Flow media from GE Healthcare Lifesciences.

Other capture columns can also be used, depending on the biologic (e.g. recombinant protein) of interest. For example, CEX, hydrophobic interaction chromatography (HIC) and/or IMAC columns can also be used as capture columns. Other capture columns also include those with antibodies specific to the biologics. In some embodiments, an affinity chromatography column may be used to capture antibodies. In some embodiments, an affinity chromatography column may be used to capture virus particles. In some embodiments, the affinity chromatography column comprises protein A column and protein Z column. In some embodiments, a size exclusion chromatography column may be used as the capture column.

The volume of the capture column (e.g. AEX column) can be any suitable volume, such as between 0.1 L and 1,000 L. Exemplary column volumes include about 0.1 L, about 0.2 L, about 0.3 L, about 0.4 L, about 0.5 L, about 0.6 L, about 0.7 L, about 0.8 L, about 0.9 L, about 1 L, about 2 L, about 3 L, about 4 L, about 5 L, about 6 L, about 7 L, about 8 L, about 9 L, about 10 L, about 20 L, about 30 L, about 40 L, about 50 L, about 60 L, about 70 L, about 80 L, about 90 L, about 100 L, about 150 L, about 200 L, about 250 L, about 300 L, about 350 L, about 400 L and about 500 L, about 600 L, about 700 L, about 800 L, about 900 L and about 1,000 L.

In one or more embodiments, the capture columns (e.g. AEX columns) are relatively small in comparison to the bioreactor size and/or the flow rate of the filtrate loaded onto the capture columns. In one or more embodiments, the ratio of the bioreactor volume to the total capture column volume is in the range of about of about 500:1 to about 10:1. Exemplary ratios include about 500:1, about 450:1, about 400:1, about 350:1, about 300:1, about 250:1, about 200:1, about 150:1, about 100:1, about 90:1, about 80:1, about 70:1, about 60:1, about 50:1, about 40:1, about 30:1, about 20:1 and about 10:1.

In one or more embodiments, the total capture column residence time (e.g. total AEX column residence time) is in the range of 0.5 minutes to 200 minutes. Exemplary total capture column residence times include 0.5 minutes, 1 minute, 1.5 minutes, 2 minutes, 2.5 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 110 minutes, 120 minutes, 130 minutes, 140 minutes, 150 minutes, 160 minutes, 170 minutes, 180 minutes, 190 minutes and 200 minutes.

In one or more embodiments, the filtrate is loaded on the at least two capture columns at a filtrate load rate in the range of about 0.5 to about 100 CV per hour. Exemplary filtrate load rates include about 0.5, about 1, about 2, about 3, about 4, about 5, about 6, about 7, about 8, about 9, about 10, about 11, about 12, about 13, about 14, about 15, about 20, about 25, about 30, about 35, about 40, about 45, about 50, about 55, about 60, about 65, about 70, about 75, about 80, about 85, about 90, about 95 and about 100 CV per hour.

In one or more embodiments, the filtrate is loaded on the at least two capture columns at a filtrate load rate in the range of about 10 to about 10,000 mL per minute. Exemplary filtrate load rates include about 10, about 11, about 12, about 13, about 14, about 15, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, about 500, about 550, about 600, about 650, about 700, about 750, about 800, about 850, about 900, about 950, about 1,000, about 1,100, about 1,200, about 1,300, about 1,400, about 1,500, about 2,000, about 2,500, about 3,000, about 3,500, about 4,000, about 4,500, about 5,000, about 6,000, about 7,000, about 8,000, about 9,000 and about 10,000 mL per minute.

Exemplary conditions for an AEX column are provided in Table 2 below:

TABLE 2

| | | Flow rate | | Temperature |
|---|---|---|---|---|
| Procedure | Buffer | (cm/h) | Volume (CV) | (° C.) |
| Pre-used | 0.1-10M NaOH | ≤25-2500 | ≥1-3 | 15-25 |
| Sanitization | | | (≥10-120 min) | |
| Pre- | 20-2000 mM phosphate | ≤25-2500 | ≥1-5 | 15-25 |
| Equilibration | buffer (PB), pH 6.9-7.3 | | | |
| Equilibration | 4-400 mM PB, pH 6.9-7.3 | ≤25-2500 | ≥1-5 | 2-25 |
| Load | NA | ≤10-1000 | NA | 2-15 |
| Wash1 | 4-400 mM PB, pH 6.9-7.3 | ≤25-2500 | ≥2-10 | 2-15 |
| Wash2 | 4-400 mM PB, pH 6.9-7.3 | ≤25-2500 | ≥2-10 | 15-25 |
| Elution | 4-400 mM PB, 20-2000 | ≤25-2500 | NA | 15-25 |
| | mM NaCl, pH 6.1-6.5 | | | |
| Strip | 4-400 mM PB, 0.1-10M | ≤25-2500 | ≥1-5 | 15-25 |
| | NaCl, pH 6.1-6.5 | | | |

After the biologics containing filtrate is loaded onto the capturing system 605, the biologic is eluted from the column(s) by changing the pH and/or salt content in the column.

The eluted biologics can be subjected to further purification steps and/or quality assurance steps. For example, as shown in FIG. 5, the eluted biologics can be subjected to a virus kill step 607. Such a virus kill 607 can include one or more of a low pH kill, a detergent kill, or other technique known in the art. In one or more embodiments, the biologic is a viral particle (e.g. AAV) that is more robust than other, non-desired viruses. In such embodiments, a virus kill step can still be performed to selectively kill the non-desired viruses.

Any of the steps shown in FIG. 5 can also be applied to the systems shown in FIGS. 6 and 7, including, but not limited to, viral kills, additional chromatography systems, additional filtering, final product adjustment, etc.

The biologics product from the virus kill step 607 can be introduced into a second chromatography system 609 to further purify the biologics product. Alternatively, the eluted biologics from the capturing system 605 can be fed directly to the second chromatography system 609. In various embodiments, the second chromatography system 609 includes one or more purification columns. In some embodiments, the purification columns comprise IMAC columns for further removal of impurities. Exemplary metal ions include cobalt, nickel, copper, iron, zinc or gallium. In some embodiments, the purification columns comprise AEX columns for further removal of impurities. In some embodiments, the purification columns comprise CEX columns for further removal of impurities. In some embodiments, the purification columns comprise affinity columns for further removal of impurities (e.g. protein A column or protein Z column). In some embodiments, the purification columns comprise size exclusion columns for further removal of impurities. In some embodiments, the purification columns comprise hydrophobic interaction chromatography (HIC) columns for further removal of impurities.

The volume of the second chromatography column (e.g. IMAC column) can be any suitable volume, such as between 0.01 L and 100 L. Exemplary column volumes include about 0.01 L, about 0.02 L, about 0.03 L, about 0.04 L, about 0.05 L, about 0.06 L, about 0.07 L, about 0.08 L, about 0.09 L, about 0.1 L, about 0.2 L, about 0.3 L, about 0.4 L, about 0.5 L, about 0.6 L, about 0.7 L, about 0.8 L, about 0.9 L, about 1 L, about 1.5 L, about 2 L, about 2.5L, about 3 L, about 3.5 L, about 4 L, about 4.5 L, about 5 L, about 6 L, about 7 L, about 8 L, about 9 L, about 10 L, about 15 L, about 20 L, about 25 L, about 30 L, about 35 L, about 40 L and about 50 L, about 60 L, about 70 L, about 80 L, about 90 L and about 100 L.

In one or more embodiments, the eluate from the capture columns is loaded on the one or more purification columns at a load rate in the range of about 10 to about 30,000 mL per minute. Exemplary purification column load rates include about 10, about 11, about 12, about 13, about 14, about 15, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, about 500, about 550, about 600, about 650, about 700, about 750, about 800, about 850, about 900, about 950, about 1,000, about 1,100, about 1,200, about 1,300, about 1,400, about 1,500, about 2,000, about 2,500, about 3,000, about 3,500, about 4,000, about 4,500, about 5,000, about 6,000, about 7,000, about 8,000, about 9,000, about 10,000, about 15,000, about 20,000, about 25,000 and about 30,00 mL per minute.

In one or more embodiments, the ratio of the bioreactor volume to the total purification column volume is in the range of about 5,000:1 to about 50:1. Exemplary ratios include about 5,000:1, about 4,500:1, about 4,000:1, about 3,500:1, about 3,000:1, about 2,500:1, about 2,000:1, about 1,500:1, about 1,000:1, about 900:1, about 800:1, about 700:1, about 600:1, about 500:1, about 400:1, about 300:1, about 200:1, about 150:1, about 100:1, about 90:1, about 80:1, about 70:1, about 60:1 and about 50:1.

In one or more embodiments, the ratio of the total capture column volume to the total purification column volume is in the range of about 20:1 to about 1:1. Exemplary ratios include about 20:1, about 15:1, about 10:1, about 9:1, about 8:1, about 7:1, about 6:1, about 5:1, about 4.5:1, about 4:1, about 3.5:1, about 3:1, about 2.5:1, about 2:1, about 1.9:1, about 1.8:1, about 1.7:1, about 1.6:1, about 1.5:1, about 1.4:1, about 1.3:1, about 1.2:1, about 1.1:1 and about 1:1.

Exemplary conditions for an IMAC column are provided in Table 3 below:

TABLE 3

| | | Flow rate | Vol |
|---|---|---|---|
| Procedure | Buffer | (cm/h) | (CV) |
| Rinse | 4-400 mM PB, pH 6.3-6.7 | ≤25-2500 | ≥1-5 |
| Pre-use | 0.01-1.0M NaOH | ≤25-2500 | ≥1-3 |
| Sanitization | | | (≥10-30 |
| | | | min) |
| Equilibration | 4-400 mM PB, pH 6.5 | ≤25-2500 | ≥1-5 |
| Wash with WFI | Water For Injection (WFI) | ≤25-2500 | ≥1-3 |
| Chelating | 0.01-1.0M Copper Acetate | ≤25-2500 | ≥1-5 |
| Wash with WFI | WFI | ≤25-2500 | ≥2-10 |
| Blank run with | 4-400 mM PB, 15-1500 mM | ≤25-2500 | ≥2-20 |
| elution buffer | Glycine, pH 6.1-6.5 | | |
| Equilibration | 4-400 mM PB, pH 6.3-6.7 | ≤25-2500 | ≥1-5 |
| Load | NA | ≤25-2500 | ≥1-5 |
| Wash1 | 4-400 mM PB, pH 6.3-6.7 | ≤25-2500 | ≥2-10 |
| Wash2 | 4-400 mM PB, 0.1-10M NaCl, | ≤25-2500 | ≥2-10 |
| | 5-30% propylene glycol, pH | | |
| | 6.3-6.7 | | |
| Wash3 | 4-400 mM PB, pH 6.3-6.7 | ≤25-2500 | ≥2-10 |
| Elution | 4-400 mM PB, 15-1500 mM | ≤25-2500 | NA |
| | Glycine, pH 6.1-6.5 | | |
| Strip | 2-200 mM PB, 5-500 mM | ≤25-2500 | ≥1-5 |
| | EDTA, 0.1-10M NaCl, pH | | |
| | 7.2-7.6 | | |

After the biologics containing filtrate is loaded onto the second chromatography system 609, the biologic is eluted from the column(s). As shown in FIG. 5, the eluted biologics can be subjected to a virus kill step 611. As with virus kill 607, virus kill 611 can include one or more of a low pH kill, a detergent kill, or other technique known in the art. In some embodiments, only one of virus kill 607 or 611 is used, or the virus kills are performed at the same stage in the purification process.

In one or more embodiments, the eluate from the second chromatography system 609 can be stored. For example, rhGAA such as ATB200 can be particularly stable in IMAC eluate. In one or more embodiments, the eluate from the second chromatography system (e.g. IMAC eluate) is stored at a temperature of 0° C. to 10° C. for a time period of 24 hours to 105 days. In one or more embodiments, the eluate from the second chromatography system is stored for up to 1, 2, 3, 4, 5, 10, 15, 20, 25, 30, 35, 40, 45, 50, 55, 60, 65, 70, 75, 80, 85, 90, 95, 100 or 105 days. In one or more embodiments, the eluate from the second chromatography system is stored at a temperature of 15° C. to 30° C. for a time period of 1 hour to 3 days.

As shown in FIG. 5, the biologics product from the virus kill step 611 can be introduced into a third chromatography system 613 to further purify the biologics product. Alternatively, the eluted biologics from the second chromatography system 609 can be fed directly to the third chromatography system 613. In various embodiments, the third chromatography system 613 includes one or more AEX columns, CEX columns, size exclusion columns, affinity columns, hydrophobic interaction chromatography columns and/or SEC columns for further removal of impurities. The biologics product is then eluted from the third chromatography system 613.

The volume of the third chromatography column (e.g. CEX or SEC column) can be any suitable volume, such as between 0.01 L and 200 L. Exemplary column volumes include about 0.01 L, about 0.02 L, about 0.03 L, about 0.04 L, about 0.05 L, about 0.06 L, about 0.07 L, about 0.08 L, about 0.09 L, about 0.1 L, about 0.2 L, about 0.3 L, about 0.4 L, about 0.5 L, about 0.6 L, about 0.7 L, about 0.8 L, about 0.9 L, about 1 L, about 1.5 L, about 2 L, about 2.5L, about 3 L, about 3.5 L, about 4 L, about 4.5 L, about 5 L, about 6 L, about 7 L, about 8 L, about 9 L, about 10 L, about 15 L, about 20 L, about 25 L, about 30 L, about 35 L, about 40 L and about 50 L, about 60 L, about 70 L, about 80 L, about 90 L, about 100 L, about 150 L and about 200 L.

Exemplary conditions for a CEX column are provided in Table 4 below:

TABLE 4

| | | Flow rate | Vol |
|---|---|---|---|
| Procedure | Buffer | (cm/h) | (CV) |
| Pre-used | 0.1-10M NaOH | ≤25-2500 | ≥1-3 |
| Sanitization | | | (≥10-120 |
| | | | min) |
| Equilibration | 2-200 mM Sodium citrate, pH | ≤30-3000 | ≥2-10 |
| | 4.0-5.0 | | |
| Load | NA | ≤30-3000 | NA |
| Wash | 2-200 mM Sodium citrate, pH | ≤30-3000 | ≥2-10 |
| | 4.0-5.0 | | |
| Elution | 2-200 mM Sodium citrate, | ≤30-3000 | ≥2-10 |
| | 15-1500 mM NaCl, pH 4.0-5.0 | | |
| Strip | 2-200 mM Sodium citrate, | ≤30-3000 | ≥1-5 |
| | 0.1-10M NaCl, pH 4.0-5.0 | | |
| Post-use | 0.1-10M NaOH | ≤25-2500 | ≥1-3 |
| Sanitization | | | (≥10-120 |
| | | | min) |
| Storage | 0.01-1.0M NaOH | ≤30-3000 | ≥1-5 |

The biologics product may also be subjected to further processing. For example, another filtration system 615 may be used to remove viruses. In some embodiments, such filtration can utilize filters with pore sizes between 5 nm and 50 μm. Other product processing can include a product adjustment step 617, in which the biologics product may be sterilized, filtered, concentrated, stored and/or have additional components for added for the final product formulation. For example, the biologics product can be concentrated by a factor of 2-10 times. This final product can be used to fill vials and may be lyophilized for future use.

### Administration of Biologics

The biologics or a pharmaceutically acceptable salt thereof, can be formulated in accordance with the routine procedures as a pharmaceutical composition adapted for administration to human beings. For example, in a preferred embodiment, a composition for intravenous administration is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

In some embodiments, the biologic (e.g. recombinant protein such as rhGAA) (or a composition or medicament containing the biologics) is administered by an appropriate route. In one embodiment, the biologic is administered intravenously. In other embodiments, biologics (e.g. rhGAA) is administered by direct administration to a target tissue, such as to heart or skeletal muscle (e.g. intramuscular), or nervous system (e.g. direct injection into the brain; intraventricularly; intrathecally). More than one route can be used concurrently, if desired.

The biologic (e.g. recombinant protein such as rhGAA) (or a composition or medicament containing the biologics) is administered in a therapeutically effective amount (e.g. a dosage amount that, when administered at regular intervals, is sufficient to treat the disease, such as by ameliorating symptoms associated with the disease, preventing or delaying the onset of the disease, and/or lessening the severity or frequency of symptoms of the disease). The amount which will be therapeutically effective in the treatment of the disease will depend on the nature and extent of the disease's effects, and can be determined by standard clinical techniques. In addition, in vitro or in vivo assays may optionally be employed to help identify optimal dosage ranges. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems. In at least one embodiment, the recombinant human acid α-glucosidase is administered by intravenous infusion at a dose of about 1 mg/kg to about 100 mg/kg, such as about 5 mg/kg to about 30 mg/kg, typically about 5 mg/kg to about 20 mg/kg. In at least one embodiment, the biologic is a recombinant protein. In some embodiments, the recombinant human acid α-glucosidase is administered by intravenous infusion at a dose of about 5 mg/kg, about 10 mg/kg, about 15 mg/kg, about 20 mg/kg, about 25 mg/kg, about 30 mg/kg, about 35 mg/kg, about 40 mg/kg, about 50 mg/kg, about 50 mg/kg, about 60 mg/kg, about 70 mg/kg, about 80 mg/kg, about 90 mg/kg or about 100 mg/kg. In at least one embodiment, the recombinant human acid α-glucosidase is administered by intravenous infusion at a dose of about 20 mg/kg. The effective dose for a particular individual can be varied (e.g. increased or decreased) over time, depending on the needs of the individual. For example, in times of physical illness or stress, or if anti-acid α-glucosidase antibodies become present or increase, or if disease symptoms worsen, the amount can be increased.

The therapeutically effective amount of recombinant human acid α-glucosidase (or composition or medicament containing recombinant human acid α-glucosidase) is administered at regular intervals, depending on the nature and extent of the disease's effects, and on an ongoing basis. Administration at a “regular interval,” as used herein, indicates that the therapeutically effective amount is administered periodically (as distinguished from a one-time dose). The interval can be determined by standard clinical techniques. In preferred embodiments, recombinant human acid α-glucosidase is administered monthly, bimonthly;

weekly; twice weekly; or daily. The administration interval for a single individual need not be a fixed interval, but can be varied over time, depending on the needs of the individual. For example, in times of physical illness or stress, if anti-recombinant human acid α-glucosidase antibodies become present or increase, or if disease symptoms worsen, the interval between doses can be decreased. In some embodiments, a therapeutically effective amount of 5, 10, 20, 50, 100, or 200 mg enzyme/kg body weight is administered twice a week, weekly or every other week with or without a chaperone.

The biologics (e.g. recombinant protein such as rhGAA) may be prepared for later use, such as in a unit dose vial or syringe, or in a bottle or bag for intravenous administration. Kits containing the biologics (e.g. recombinant protein such as rhGAA), as well as optional excipients or other active ingredients, such as chaperones or other drugs, may be enclosed in packaging material and accompanied by instructions for reconstitution, dilution or dosing for treating a subject in need of treatment, such as a patient having Pompe disease.

Combination Therapy of rhGAA and Pharmacological Chaperone

In various embodiments, the rhGAA (e.g. ATB200) produced by the processes described herein can be used in combination therapy with a pharmacological chaperone such as miglustat or duvoglustat.

In at least one embodiment, the pharmacological chaperone (e.g. miglustat) is administered orally. In at least one embodiment, the miglustat is administered at an oral dose of about 200 mg to about 400 mg, or at an oral dose of about 200 mg, about 250 mg, about 300 mg, about 350 mg or about 400 mg. In at least one embodiment, the miglustat is administered at an oral dose of about 233 mg to about 400 mg. In at least one embodiment, the miglustat is administered at an oral dose of about 250 to about 270 mg, or at an oral dose of about 250 mg, about 255 mg, about 260 mg, about 265 mg or about 270 mg. In at least one embodiment, the miglustat is administered as an oral dose of about 260 mg.

It will be understood by those skilled in the art that an oral dose of miglustat in the range of about 200 mg to 400 mg or any smaller range therewithin can be suitable for an adult patient with an average body weight of about 70 kg. For patients having a significantly lower body weight than about 70 kg, including but not limited to infants, children or underweight adults, a smaller dose may be considered suitable by a physician. Therefore, in at least one embodiment, the miglustat is administered as an oral dose of from about 50 mg to about 200 mg, or as an oral dose of about 50 mg, about 75 mg, about 100 mg, 125 mg, about 150 mg, about 175 mg or about 200 mg. In at least one embodiment, the miglustat is administered as an oral dose of from about 65 mg to about 195 mg, or as an oral dose of about 65 mg, about 130 mg or about 195 mg.

In at least one embodiment, the miglustat is administered as a pharmaceutically acceptable dosage form suitable for oral administration, and includes but is not limited to tablets, capsules, ovules, elixirs, solutions or suspensions, gels, syrups, mouth washes, or a dry powder for reconstitution with water or other suitable vehicle before use, optionally with flavoring and coloring agents for immediate-, delayed-, modified-, sustained-, pulsed- or controlled-release applications. Solid compositions such as tablets, capsules, lozenges, pastilles, pills, boluses, powder, pastes, granules, bullets, dragees or premix preparations can also be used. In at least one embodiment, the miglustat is administered as a tablet. In at least one embodiment, the miglustat is administered as a capsule. In at least one embodiment, the dosage form contains from about 50 mg to about 300 mg of miglustat. In at least one embodiment, the dosage form contains about 65 mg of miglustat. In at least one embodiment, the dosage form contains about 130 mg of miglustat. In at least one embodiment, the dosage form contains about 260 mg of miglustat. It is contemplated that when the dosage form contains about 65 mg of miglustat, the miglustat can be administered as a dosage of four dosage forms, or a total dose of 260 mg of miglustat. However, for patients who have a significantly lower weight than an average adult weight of 70 kg, including but not limited to infants, children or underweight adults, the miglustat can be administered as a dosage of one dosage form (a total dose of 65 mg of miglustat), two dosage forms (a total dose of 130 mg of miglustat), or three dosage forms (a total dose of 195 mg of miglustat).

Solid and liquid compositions for oral use can be prepared according to methods well known in the art. Such compositions can also contain one or more pharmaceutically acceptable carriers and excipients which can be in solid or liquid form. Tablets or capsules can be prepared by conventional means with pharmaceutically acceptable excipients, including but not limited to binding agents, fillers, lubricants, disintegrants or wetting agents. Suitable pharmaceutically acceptable excipients are known in the art and include but are not limited to pregelatinized starch, polyvinylpyrrolidone, povidone, hydroxypropyl methylcellulose (HPMC), hydroxypropyl ethylcellulose (HPEC), hydroxypropyl cellulose (HPC), sucrose, gelatin, acacia, lactose, microcrystalline cellulose, calcium hydrogen phosphate, magnesium stearate, stearic acid, glyceryl behenate, talc, silica, corn, potato or tapioca starch, sodium starch glycolate, sodium lauryl sulfate, sodium citrate, calcium carbonate, dibasic calcium phosphate, glycine croscarmellose sodium and complex silicates. Tablets can be coated by methods well known in the art. In at least one embodiment, the miglustat is administered as a formulation available commercially as Zavesca® (Actelion Pharmaceuticals).

In at least one embodiment, the miglustat and the recombinant human acid α-glucosidase are administered simultaneously. In at least one embodiment, the miglustat and the recombinant human acid α-glucosidase are administered sequentially. In at least one embodiment, the miglustat is administered prior to administration of the recombinant human acid α-glucosidase. In at least one embodiment, the miglustat is administered less than three hours prior to administration of the recombinant human acid α-glucosidase. In at least one embodiment, the miglustat is administered about two hours prior to administration of the recombinant human acid α-glucosidase. In at least one embodiment, the miglustat is administered less than two hours prior to administration of the recombinant human acid α-glucosidase. In at least one embodiment, the miglustat is administered about 1.5 hours prior to administration of the recombinant human acid α-glucosidase. In at least one embodiment, the miglustat is administered about one hour prior to administration of the recombinant human acid α-glucosidase. In at least one embodiment, the miglustat is administered from about 50 minutes to about 70 minutes prior to administration of the recombinant human acid α-glucosidase. In at least one embodiment, the miglustat is administered from about 55 minutes to about 65 minutes prior to administration of the recombinant human acid α-glucosidase. In at least one embodiment, the miglustat is administered about 30 minutes prior to administration of the recombinant human acid α-glucosidase. In at least one embodiment, the miglustat is administered from about 25 minutes to about 35 minutes prior to administration of the recombinant human acid α-glucosidase. In at least one embodiment, the miglustat is administered from about 27 minutes to about 33 minutes prior to administration of the recombinant human acid α-glucosidase.

In at least one embodiment, the miglustat is administered concurrently with administration of the recombinant human acid α-glucosidase. In at least one embodiment, the miglustat is administered within 20 minutes before or after administration of the recombinant human acid α-glucosidase. In at least one embodiment, the miglustat is administered within 15 minutes before or after administration of the recombinant human acid α-glucosidase. In at least one embodiment, the miglustat is administered within 10 minutes before or after administration of the recombinant human acid α-glucosidase. In at least one embodiment, the miglustat is administered within 5 minutes before or after administration of the recombinant human acid α-glucosidase.

In at least one embodiment, the miglustat is administered after administration of the recombinant human acid α-glucosidase. In at least one embodiment, the miglustat is administered up to 2 hours after administration of the recombinant human acid α-glucosidase. In at least one embodiment, the miglustat is administered about 30 minutes after administration of the recombinant human acid α-glucosidase. In at least one embodiment, the miglustat is administered about one hour after administration of the recombinant human acid α-glucosidase. In at least one embodiment, the miglustat is administered about 1.5 hours after administration of the recombinant human acid α-glucosidase. In at least one embodiment, the miglustat is administered about 2 hours after administration of the recombinant human acid α-glucosidase.

Another aspect of the invention provides a kit for combination therapy of Pompe disease in a patient in need thereof. The kit includes a pharmaceutically acceptable dosage form comprising miglustat, a pharmaceutically acceptable dosage form comprising a recombinant human acid α-glucosidase as defined herein, and instructions for administering the pharmaceutically acceptable dosage form comprising miglustat and the pharmaceutically acceptable dosage form comprising the recombinant acid α-glucosidase to a patient in need thereof. In at least one embodiment, the pharmaceutically acceptable dosage form comprising miglustat is an oral dosage form as described herein, including but not limited to a tablet or a capsule. In at least one embodiment, the pharmaceutically acceptable dosage form comprising a recombinant human acid α-glucosidase is a sterile solution suitable for injection as described herein. In at least one embodiment, the instructions for administering the dosage forms include instructions to administer the pharmaceutically acceptable dosage form comprising miglustat orally prior to administering the pharmaceutically acceptable dosage form comprising the recombinant human acid α-glucosidase by intravenous infusion, as described herein.

Without being bound by theory, it is believed that miglustat acts as a pharmacological chaperone for the recombinant human acid α-glucosidase ATB200 and binds to its active site. For example, miglustat has been found to decrease the percentage of unfolded ATB200 protein and stabilize the active conformation of ATB200, preventing denaturation and irreversible inactivation at the neutral pH of plasma and allowing it to survive conditions in the circulation long enough to reach and be taken up by tissues. However, the binding of miglustat to the active site of ATB200 also can result in inhibition of the enzymatic activity of ATB200 by preventing the natural substrate, glycogen, from accessing the active site. It is believed that when miglustat and the recombinant human acid α-glucosidase are administered to a patient under the conditions described herein, the concentrations of miglustat and ATB200 within the plasma and tissues are such that ATB200 is stabilized until it can be taken up into the tissues and targeted to lysosomes, but, because of the rapid clearance of miglustat, hydrolysis of glycogen by ATB200 within lysosomes is not overly inhibited by the presence of miglustat, and the enzyme retains sufficient activity to be therapeutically useful.

All the embodiments described above may be combined. This includes in particular embodiments relating to:

the nature of the pharmacological chaperone, for example miglustat; and the active site for which it is specific;

the dosage, route of administration of the pharmacological chaperone (e.g. miglustat) and the type of pharmaceutical composition including the nature of the carrier and the use of commercially available compositions;

the nature of the drug, e.g. therapeutic protein drug product, which may be a counterpart of an endogenous protein for which expression is reduced or absent in the subject, suitably recombinant protein (e.g. rhGAA), for example the recombinant human acid α-glucosidase expressed in Chinese hamster ovary (CHO) cells and comprising an increased content of N-glycan units bearing one or more mannose-6-phosphate residues when compared to a content of N-glycan units bearing one or more mannose-6-phosphate residues of alglucosidase alfa; and suitably having an amino acid sequence as set forth in SEQ ID NO: 1 or SEQ ID NO: 2;

the number and type of N-glycan units on the recombinant protein (e.g. rhGAA), such as the N-acetylglucosamine, galactose, sialic acid or complex N-glycans formed from combinations of these, attached to the recombinant protein;

the degree of phosphorylation of mannose units on the recombinant protein (e.g. rhGAA) to form mannose-6-phosphate and/or bis-mannose-6-phosphate;

the dosage and route of administration (e.g. intravenous administration, especially intravenous infusion, or direct administration to the target tissue) of the replacement enzyme (e.g. recombinant human acid α-glucosidase) and the type of formulation including carriers and therapeutically effective amount;

the dosage interval of the pharmacological chaperone (miglustat) and the recombinant human acid α-glucosidase;

the nature of the therapeutic response and the results of the combination therapy (e.g. enhanced results as compared to the effect of each therapy performed individually);

the timing of the administration of the combination therapy, e.g. simultaneous administration of miglustat and the recombinant human acid α-glucosidase or sequential administration, for example wherein the miglustat is administered prior to the recombinant human acid α-glucosidase or after the recombinant human acid α-glucosidase or within a certain time before or after administration of the recombinant human acid α-glucosidase; and

the nature of the patient treated (e.g. mammal such as human) and the condition suffered by the individual (e.g. enzyme insufficiency).

Any of the embodiments in the list above may be combined with one or more of the other embodiments in the list.

### EXAMPLES

Other features of the present invention will become apparent from the following non-limiting examples which illustrate, by way of example, the principles of the invention.

### Example 1

### Preparation of CHO Cells Producing ATB200 RhGAA having a High Content of Mono- or Bis-M6P-Bearing N-Glycans

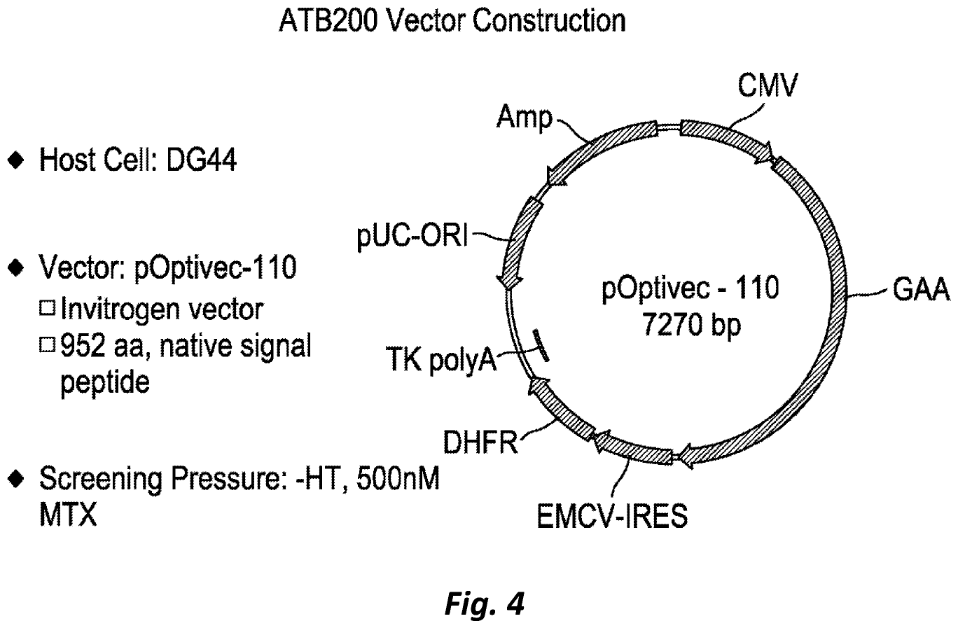

CHO cells were transfected with DNA that expresses rhGAA followed by selection of transformants producing rhGAA. A DNA construct for transforming CHO cells with DNA encoding rhGAA is shown in FIG. 4. CHO cells were transfected with DNA that expresses rhGAA followed by selection of transformants producing rhGAA.

After transfection, DG44 CHO (DHFR-) cells containing a stably integrated GAA gene were selected with hypoxanthine/thymidine deficient (-HT) medium). Amplification of

GAA expression in these cells was induced by methotrexate treatment (MTX, 500 nM). Cell pools that expressed high amounts of GAA were identified by GAA enzyme activity assays and were used to establish individual clones producing rhGAA. Individual clones were generated on semisolid media plates, picked by ClonePix system, and were transferred to 24-deep well plates. The individual clones were assayed for GAA enzyme activity to identify clones expressing a high level of GAA. Conditioned media for determining GAA activity used a 4-MU-α-Glucosidase substrate. Clones producing higher levels of GAA as measured by GAA enzyme assays were further evaluated for viability, ability to grow, GAA productivity, N-glycan structure and stable protein expression. CHO cell lines, including CHO cell line GA-ATB-200, expressing rhGAA with enhanced mono-M6P or bis-M6P N-glycans were isolated using this procedure.

### Example 2

### Proof-of-Concept Plan for Capturing and Purification of ATB200 RhGAA

Cells expressing ATB200 rhGAA are cultured in a bioreactor. Cell media is removed, filtered and frozen for later use. A bulk container with the thawed harvest is then used in batch mode to load two AEX columns that each have a column volume 15.7 mL (1 cm diameter x 20 cm bed height). The AEX columns are loaded at a flow rate of 1.57 mL/min. The total AEX column residence time (i.e. the quotient of the total AEX column volume and the volumetric flow rate loading the AEX columns) is 20 minutes.

The continuous process was run according to the following protocol:
Execute two sequences (equates to the same number of runs as the control conditions)
Each AEX eluate will be continuously processed over the IMAC column
   i. Sequence 1
      1. Load AEX1→STP AEX1 eluate onto IMAC→Collect IMAC eluate (IMACa)
      2. Load AEX2→STP AEX2 eluate onto IMAC→Collect IMAC eluate (IMACb)
   ii. Sequence 2
      1. Load AEX1→STP AEX1 eluate onto IMAC→Collect IMAC eluate (IMACa)
      2. Load AEX2→STP AEX2 eluate onto IMAC→Collect IMAC eluate (IMACb)

A batch process was also run as a control according to the following protocol:
Execute four runs (two runs on each AEX column: AEX1, AEX2)
Each AEX eluate will be collected and processed over the IMAC column
   i. Control Run 1=Load AEX1→Collect AEX1 eluate→Load AEX1 eluate onto IMAC→Collect IMAC eluate
   ii. Control Run 2=Load AEX2→Collect AEX2 eluate→Load AEX2 eluate onto IMAC→Collect IMAC eluate
   iii. Control Run 3=Load AEX1→Collect AEX1 eluate→Load AEX1 eluate onto IMAC Collect IMAC eluate
   iv. Control Run 4=Load AEX2→Collect AEX2 eluate→Load AEX2 eluate onto IMAC→Collect IMAC eluate

The process conditions for each AEX column are provided in Table 5 below:

TABLE 5

| | | | Flow | Flow | Flow | | |
|---|---|---|---|---|---|---|---|
| | | | Rate | Rate | Rate | Residence | Target |
| | | Flow | LFR | VFR | VFR | Time | CV or |
| Buffer/Solution | Step | Direction | (cm/hr) | (mL/min) | (L/hr) | (min) | min |
| 1M NaOH | Pre-Use | Down | 201.7 | 2.64 | 0.16 | 5.9 | ≥2 CV |
| | Sanitization | | 80.2 | 1.05 | 0.06 | 15.0 | |
| 200 mM PB | Pre-Use | Down | 201.7 | 2.64 | 0.16 | 5.9 | ≥3 CV |
| pH 7.1 | Equilibration | | | | | | |
| 40 mM PB | Equilibration | Down | 201.7 | 2.64 | 0.16 | 5.9 | ≥3 CV |
| pH 7.1 | | | | | | | |
| ATF Filtrate | Load | Down | 119.9 | 1.57 | 0.09 | 10.0 | — |
| 40 mM PB | Wash 1 | Down | 201.7 | 2.64 | 0.16 | 5.9 | ≥5 CV |
| pH 7.1 | | | | | | | |
| 40 mM PB | Wash 2 | Down | 201.7 | 2.64 | 0.16 | 5.9 | ≥5 CV |
| pH 6.3 | | | | | | | |
| 40 mM PB, | Elution | Down | 49.7 | 0.65 | 0.04 | 24.2 | CVfor |
| 180 mM NaCl | | | | | | | eluate |
| pH 6.3 | | | | | | | collection |
| 40 mM PB, 1M | Strip | Up | 201.7 | 2.64 | 0.16 | 5.9 | ≥3 CV |
| NaCl pH 6.3 | | | | | | | |

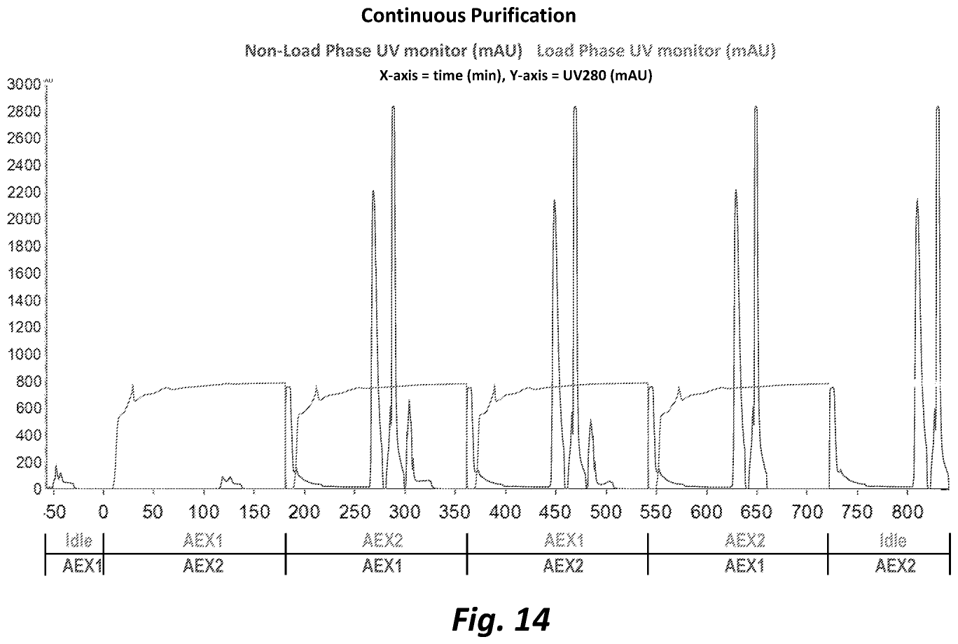

The elution profile for the control process and continuous process are shown in FIGS. 13 and 14, respectively.

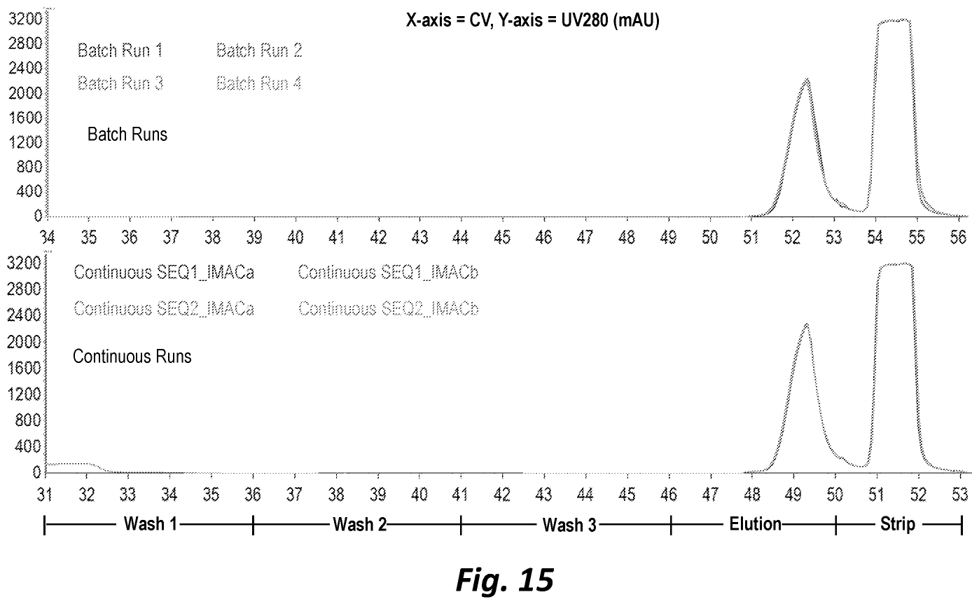

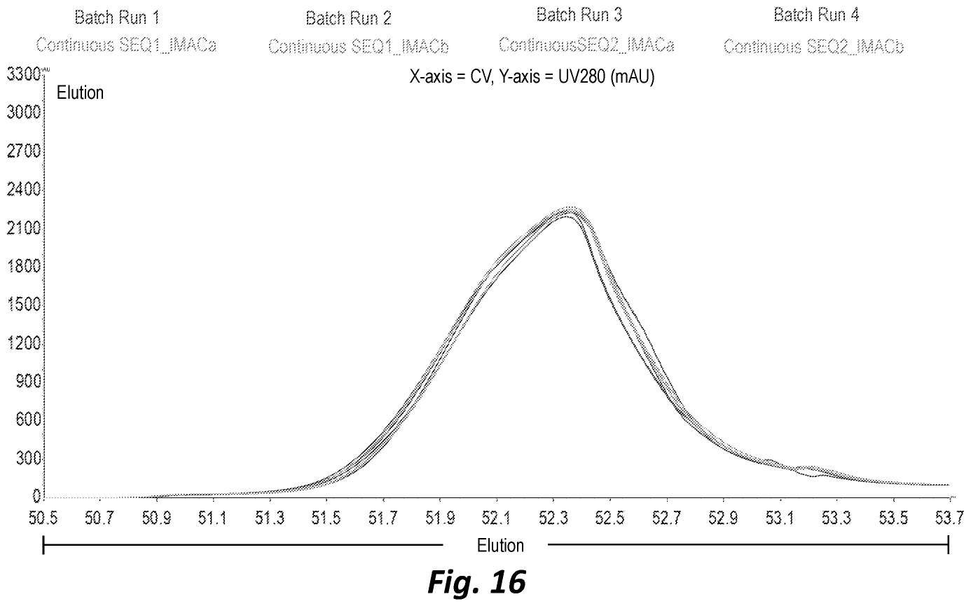

The AEX eluate is then loaded on a single IMAC column having a column volume of 3.9 mL (1 cm diameter×5 cm bed height). The IMAC column is loaded at a flow rate of 0.65 mL/min. The ratio of the total AEX column volume to the IMAC column volume is approximately 8:1. The elution profile for both the batch purification process and the continuous purification process was recorded, which is shown in FIGS. 15 and 16. The process conditions for the IMAC column are provided in Table 6 below:

TABLE 6

| | | | Flow | Flow | Flow | | |
|---|---|---|---|---|---|---|---|
| | | | Rate | Rate | Rate | Residence | Target |
| | | Flow | LFR | VFR | VFR | Time | CV or |
| Buffer/Solution | Step | Direction | (cm/hr) | (mL/min) | (L/hr) | (min) | min |
| 1M NaOH | Pre-Use | Down | 249.8 | 3.27 | 0.20 | 1.2 | ≥2 CV |
| | Sanitization | | 19.9 | 0.26 | 0.02 | 15.1 | |
| 40 mM PB | Equilibration | Down | 249.8 | 3.27 | 0.20 | 1.2 | ≥3 CV |
| pH 6.5 | | | | | | | |
| WFI | Rinse | Down | 249.8 | 3.27 | 0.20 | 1.2 | ≥3 CV |
| 0.1M Copper | Chelating | Down | 49.7 | 0.65 | 0.04 | 6.0 | ≥ 1 CV |
| Acetate | | | | | | | |
| WFI | Rinse | Down | 249.8 | 3.27 | 0.20 | 1.2 | ≥3 CV |
| 40 mM PB, | Blank | Down | 249.8 | 3.27 | 0.20 | 1.2 | ≥5 CV |
| 150 mM | Elution | | 49.7 | 0.65 | 0.04 | 6.0 | |
| Glycine pH 6.3 | | | | | | | |
| 40 mM PB | Equilibration | Down | 249.8 | 3.27 | 0.20 | 1.2 | ≥3 CV |
| pH 6.5 | | | | | | | |
| AEX Eluate | Load | Down | 49.7 | 0.65 | 0.04 | 6.0 | — |
| 40 mM PB | Wash 1 | Down | 249.8 | 3.27 | 0.20 | 1.2 | ≥5 CV |
| pH 6.5 | | | | | | | |
| 40 mM PB, 1M | Wash 2 | Down | 249.8 | 3.27 | 0.20 | 1.2 | ≥5 CV |
| NaCl, 10% | | | | | | | |
| Propylene Glycol | | | | | | | |
| pH 6.5 | | | | | | | |
| 40 mM PB | Wash 3 | Down | 249.8 | 3.27 | 0.20 | 1.2 | ≥5 CV |
| pH 6.5 | | | | | | | |
| 40 mM PB, | Elution | Down | 49.7 | 0.65 | 0.04 | 6.0 | CVfor |
| 150 mM Glycine | | | | | | | eluate |
| pH 6.3 | | | | | | | collection |
| 20 mM PB, 0.5M | Strip | Down | 249.8 | 3.27 | 0.20 | 1.2 | ≥3 CV |
| NaCl, 50 mM | | | | | | | |
| EDTA pH 7.4 | | | | | | | |

The rhGAA produced according to the continuous process according to this example was comparable to rhGAA produced according to a control (previous batch mode) process, thus showing no loss of product quality using the continuous process in this Example 2. However, the proposed facility footprint implementing the continuous process of this Example 2 is estimated to provide a four to five times reduction in facility footprint.

The final products from batch purification and the continuous purification were anlalyze. The data is listed in table 7-9.

TABLE 7

| | | | | | | | | AEX + |
|---|---|---|---|---|---|---|---|---|
| | | | | | Sialic | | | IMAC |
| | Enzyme | Specific | | | Acid | M6P | Bis- | Activity |
| | Activity | Activity | HCP | SEC (% | (mol/mol | (mol/mol | M6P | Recovery |
| Run | (nmol/mL/hr) | (nmol/mg/hr) | (ppm) | Monomer) | ATB200) | ATB200) | (%) | (%)^{a} |
| Batch 1 | 421,990 | 109,019 | 7,555 | 95.4 | 6.3 | 3.93 | 18.19 | 51.6 |
| Batch 2 | 434,599 | 107,864 | 7,444 | 95.9 | 6.3 | 3.77 | 17.01 | 58.1 |
| Batch 3 | 395,123 | 105,881 | 7,858 | 95.4 | 6.1 | 4.00 | 17.20 | 55.3 |
| Batch 4 | 409,479 | 106,964 | 7,586 | 95.8 | 6.2 | 3.77 | 17.18 | 55.3 |
| Cont. | 429,035 | 108,996 | 7,690 | 95.5 | 6.2 | 4.05 | 18.05 | 56.4 |
| SEQ1a | | | | | | | | |
| Cont. | 444,879 | 110,077 | 7,674 | 95.8 | 6.3 | 3.79 | 16.78 | 57.5 |
| SEQ1b | | | | | | | | |
| Cont. | 427,765 | 109,693 | 7,845 | 95.6 | 6.3 | 3.82 | 17.29 | 54.1 |
| SEQ2a | | | | | | | | |
| Cont. | 432,513 | 108,135 | 7,549 | 95.8 | 6.3 | 3.68 | 16.28 | 57.3 |
| SEQ2b | | | | | | | | |

^{a}Activity recovery is the total process recovery through both the AEX and IMAC unit operations.

TABLE 8

| | Enzyme Activity | | Specific Activity | | HCP | | SEC | |
|---|---|---|---|---|---|---|---|---|
| | (nmol/mL/hr) | | (nmol/mg/hr) | | (ppm) | | (% Monomer) | |
| | Batch | Continuous | Batch | Continuous | Batch | Continuous | Batch | Continuous |
| Mean | 415,298 | 433,548 | 107,432 | 109,225 | 7,611 | 7,690 | 95.6 | 95.7 |
| Std Dev | 16,914 | 7,816 | 1,333 | 854 | 176 | 121 | 0.26 | 0.15 |
| Min | 395,123 | 427,765 | 105,881 | 108,135 | 7,444 | 7,549 | 95.4 | 95.5 |
| Max | 434,599 | 444,879 | 109,019 | 110,077 | 7,858 | 7,845 | 95.9 | 95.8 |
| Range | 39,476 | 17,114 | 3,138 | 1,942 | 414 | 296 | 0.5 | 0.3 |
| N | 4 | 4 | 4 | 4 | 4 | 4 | 4 | 4 |

TABLE 9

| | Sialic Acid | | M6P | | | | AEX + IMAC | |
|---|---|---|---|---|---|---|---|---|
| | (mol/mol | | (mol/mol | | Bis-M6P | | Activity | |
| | ATB200) | | ATB200) | | (%) | | Recovery (%) | |
| | Batch | Continuous | Batch | Continuous | Batch | Continuous | Batch | Continuous |
| Mean | 6.2 | 6.3 | 3.87 | 3.84 | 17.4 | 17.1 | 55.1 | 56.3 |
| Std Dev | 0.10 | 0.05 | 0.12 | 0.16 | 0.54 | 0.76 | 2.67 | 1.56 |
| Min | 6.1 | 6.2 | 3.77 | 3.68 | 17.0 | 16.3 | 51.6 | 54.1 |
| Max | 6.3 | 6.3 | 4.00 | 4.05 | 18.2 | 18.1 | 58.1 | 57.5 |
| Range | 0.2 | 0.1 | 0.23 | 0.37 | 1.2 | 1.8 | 6.5 | 3.4 |
| N | 4 | 4 | 4 | 4 | 4 | 4 | 4 | 4 |

### Example 3

### Commercial-Scale Plant for Capturing and Purification of ATB200 RhGAA

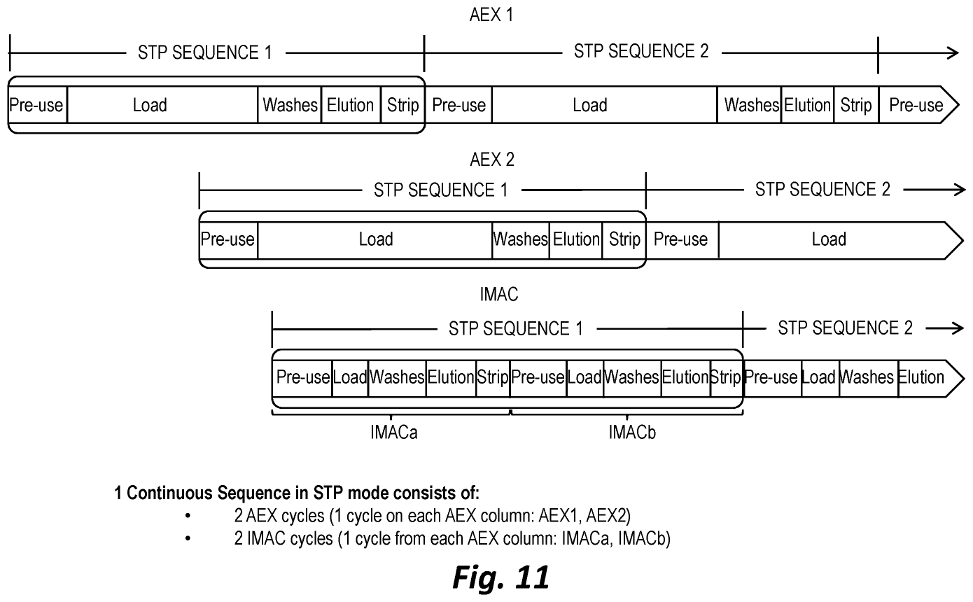

Cells expressing ATB200 rhGAA are cultured in a large bioreactor (e.g. 500-2,000 L). Cell media is continuously removed, filtered and loaded onto two AEX columns that each have a column volume of 5-25 L. The AEX columns are arranged as capture columns shown in FIG. 6. The ratio of the bioreactor volume to total AEX column volume is in the range of about 100:1 to about 20:1. The AEX eluate is directly loaded onto an IMAC column. The IMAC column volume is 1-10 L and the ratio of the total AEX column volume to the IMAC column volume is 2:1 to 10:1. FIG. 11 describes the exemplary working sequences for AEX columns and IMAC column in purifying rhGAA.

The embodiments described herein are intended to be illustrative of the present compositions and methods and are not intended to limit the scope of the present invention.

Various modifications and changes consistent with the description as a whole and which are readily apparent to the person of skill in the art are intended to be included. The appended claims should not be limited by the specific embodiments set forth in the examples, but should be given the broadest interpretation consistent with the description as a whole.

Patents, patent applications, publications, product descriptions, GenBank Accession Numbers, and protocols are cited throughout this application, the disclosures of which are incorporated herein by reference in their entireties for all purposes.

1. A method for manufacturing biologics, the method comprising:
culturing host cells in a bioreactor that produce biologics and optionally secretes the biologics;
removing media and/or cell suspension from the bioreactor;
processing the media and/or cell suspension to separate a filtrate containing the biologics;
loading the filtrate onto at least two capture columns to capture the biologics;
eluting a first biologic product from the at least two capture columns;
loading the first biologic product onto one or more purification columns; and
eluting a second biologic product from the one or more purification columns;
wherein the bioreactor has a bioreactor volume, the at least two capture columns have a total capture column volume, and wherein the ratio of the bioreactor volume to the total capture column volume is in the range of about 500:1 to about 10:1.

2. The method of claim 1, wherein the biologics comprise one or more of a recombinant protein, a virus particle or an antibody.

3. The method of claim 2, wherein the recombinant protein is a secreted protein, a membrane protein or an intracellular protein produced by the host cells.

4. (canceled)

5. (canceled)

6. The method of claim 1, wherein the at least two capture columns are loaded sequentially to provide continuous loading of the filtrate onto the at least two capture columns.

7. The method of claim 1, wherein the filtrate is loaded on the at least two capture columns at a filtrate load rate in the range of about 0.5 to about 100 column volumes (CV) per hour.

8. The method of claim 1, wherein the filtrate is loaded on the at least two capture columns to provide a capture column load time of less than 48 hours for each capture column.

9. The method of claim 1, wherein the biologics comprise recombination human lysosomal protein.

10. The method of claim 1, wherein the at least two capture columns comprise at least two anion exchange chromatography (AEX) columns, at least two affinity chromatography columns, at least two cation exchange chromatography (CEX) columns, at least two immobilized metal affinity chromatography (IMAC) columns, at least two size exclusion chromatography (SEC) columns or at least two hydrophobic interaction chromatography (HIC) columns.

11. The method of claim 1, wherein the at least two capture columns comprise at least two AEX columns.

12. The method of claim 10, wherein the affinity chromatography column comprises one or more of a protein A column and a protein Z column.

13. (canceled)

14. (canceled)

15. (canceled)

16. (canceled)

17. The method of claim 1, wherein the one or more purification columns comprise one or more anion exchange chromatography (AEX) columns, one or more affinity chromatography columns, one or more cation exchange chromatography (CEX) columns, one or more immobilized metal affinity chromatography (IMAC) columns, one or more size exclusion chromatography (SEC) columns or one or more hydrophobic interaction chromatography (HIC) columns.

18. (canceled)

19. (canceled)

20. (canceled)

21. (canceled)

22. (canceled)

23. (canceled)

24. The method of claim 1, wherein the second biologic product is eluted from the one or more purification columns within 48 hours of removing the media and/or cell suspension from the bioreactor.

25. The method of claim 1, wherein the one or more purification columns have a total purification column volume and the ratio of the bioreactor volume to the total purification column volume is in the range of about 5,000:1 to about 50:1.

26. The method of claim 1, wherein ratio of the total capture column volume to the total purification column volume is in the range of about 20:1 to about 1:1.

27. A method for manufacturing recombinant human lysosomal proteins, the method comprising:
culturing host cells in a bioreactor that produce a recombinant human lysosomal protein and optionally secrete the recombinant human lysosomal protein;
removing media and/or cell suspension from the bioreactor;
processing the media and/or cell suspension to seperate a filtrate containing the lysosomal protein;
loading the filtrate onto at least two anion exchange chromatography (AEX) columns to capture the lysosomal protein;
eluting a first biologic product from the at least two AEX columns;
loading the first biologic product onto one or more immobilized metal affinity chromatography (IMAC) columns; and
eluting a second biologic product from the one or more IMAC columns;
wherein the bioreactor has a bioreactor volume, the at least two AEX columns have a total AEX column volume, and wherein the ratio of the bioreactor volume to the total AEX column volume is in the range of about 500:1 to about 10:1.

28. (canceled)

29. (canceled)

30. (canceled)

31. The method of claim 27, wherein the at least two AEX columns are loaded sequentially to provide continuous loading of the filtrate onto the at least two AEX columns.

32. The method of claim 31, wherein the filtrate is loaded on the at least two AEX columns at a filtrate load rate in the range of about 0.5 to about 100 column volumes (CV) per hour.

33. The method of claim 32, wherein the filtrate is loaded on the at least two AEX columns to provide an AEX load time of less than 48 hours for each AEX column.

34. The method of claim 33, wherein each AEX column has a column volume of less than or equal to 50 L.

35-53. (canceled)

54. A biologic product manufactured by the method of claim 1.

55-60. (canceled)


SEQUENCE LISTING (2 sequences)

SEQ ID NO: 1 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 2 - protein, 896 aa, Homo sapiens
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC